SEQUENCE LISTING (8 sequences)

SEQ ID NO: 1 - protein, 473 aa, Artificial Sequence
  heavy chain of conatumumab IgG1
MACPGFLWALVISTCLEFSMAQVQLQESGPGLVKPSQTLSLTCTVSGGSISSGDYFWSWIRQLPGKGLEWIGHIHNSGTTYYNPSLKSRVTISVDTSKKQFSLRLSSVTAADTAVYYCARDRGGDYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 483 aa, Artificial Sequence
  heavy chain of conatumumab IgA2
MACPGFLWALVISTCLEFSMAQVQLQESGPGLVKPSQTLSLTCTVSGGSISSGDYFWSWIRQLPGKGLEWIGHIHNSGTTYYNPSLKSRVTISVDTSKKQFSLRLSSVTAADTAVYYCARDRGGDYYYGMDVWGQGTTVTVSSASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 3 - protein, 236 aa, Artificial Sequence
  kappa light chain of conatumumab
MACPGFLWALVISTCLEFSMAEIVLTQSPGTLSLSPGERATLSCRASQGISRSYLAWYQQKPGQAPSLLIYGASSRATGIPDRFSGSGSGTDFTLTISRLEPEDFAVYYCQQFGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 187 aa, Artificial Sequence
  J chain precursor
MKNHLLFWGVLAVFIKAVHVKAQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPDLESRGPFEQKLISEEDLNMHTGHHHHHH

SEQ ID NO: 5 - DNA, 1422 bp, Artificial Sequence
  heavy chain of conatumumab IgG1
atggcctgtcctggatttctgtgggccctcgtgatctctacctgcctggaattcagcatggcccaggttcagctgcaagagtctggacctggcctggtcaagccctctcagaccctgtctctgacctgtacagtgtccggcggctccatctcttccggcgactacttctggtcctggatcagacagctgcccggcaaaggcctggaatggatcggccacatccacaactctggcaccacctactacaaccccagcctgaagtccagagtgaccatctccgtggacacctccaagaagcagttctccctgcggctgtcctctgtgaccgctgctgataccgccgtgtactactgcgccagagatagaggcggcgattactactacggcatggacgtgtggggccagggcacaacagtgacagtctcttccgctagcaccaagggaccctctgtgtttcctctggctccctccagcaagtctacctctggtggaacagctgccctgggctgcctggtcaaggattactttcctgagcctgtgaccgtgtcctggaactctggcgctctgacatctggcgtgcacacctttccagctgtgctgcagtcctccggcctgtactctctgtcctctgtcgtgaccgtgccttccagctctctgggcacccagacctacatctgcaatgtgaaccacaagccttccaacaccaaggtggacaagaaggtggaacccaagtcctgcgacaagacccacacctgtcctccatgtcctgctccagaactgctcggcggaccttccgtgttcctgtttcctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccatctcgggacgagctgaccaagaaccaggtgtccctgacctgtctcgtgaagggcttctacccctccgacattgccgtggaatgggagtctaatggccagcctgagaacaactacaagacaacccctcctgtgctggactccgacggctcattcttcctgtactccaagctgacagtggacaagtccagatggcagcagggcaacgtgttctcctgctccgtgatgcacgaggccctgcacaatcactacacacagaagtccctgtctctgtcccctggcaagtaa

SEQ ID NO: 6 - DNA, 1452 bp, Artificial Sequence
  heavy chain of conatumumab IgA2
atggcctgtcctggatttctgtgggccctcgtgatctctacctgcctggaattcagcatggcccaggttcagctgcaagagtctggacctggcctggtcaagccctctcagaccctgtctctgacctgtacagtgtccggcggctccatctcttccggcgactacttctggtcctggatcagacagctgcccggcaaaggcctggaatggatcggccacatccacaactctggcaccacctactacaaccccagcctgaagtccagagtgaccatctccgtggacacctccaagaagcagttctccctgcggctgtcctctgtgaccgctgctgataccgccgtgtactactgcgccagagatagaggcggcgattactactacggcatggacgtgtggggccagggcacaacagtgacagtctcttccgctagccctacctctcctaaggtgttccctctgagcctggacagcacccctcaggatggaaatgtggtggtggcctgtctggtgcagggattcttcccacaagagcccctgtccgtgacttggagcgagtctggacagaacgtgaccgccagaaacttcccaccttctcaggacgcctctggcgacctgtacaccacctcttctcagctgaccctgcctgccacacagtgccctgatggcaagtctgtgacctgccacgtgaagcactacaccaatcctagccaggacgtgaccgtgccttgtcctgttcctcctccacctccttgctgtcaccctcggctgtctctgcacagacccgctctggaagatctgctgctgggctctgaggccaacctgacatgtaccctgaccggcctgagagatgcttctggcgccacctttacctggacaccttccagcggaaagtccgctgttcagggacctcctgagagggacctgtgcggctgttactctgtgtcctctgtgctgcctggctgtgcccagccttggaatcacggcgagacattcacctgtaccgctgctcaccccgagctgaaaacccctctgaccgccaacatcaccaagtccggcaacaccttccggcctgaagtgcatctgctgcctccaccttccgaggaactggccctgaatgagctggtcaccctgacctgtctggccaggggctttagccctaaggacgtgctcgttagatggctgcagggctcccaagagctgcccagagagaagtatctgacctgggcctctcggcaagagccatctcagggcaccacaacctttgccgtgaccagcatcctgagagtggccgccgaagattggaagaagggcgacaccttcagctgcatggtcggacatgaagccctgcctctggctttcacccagaaaaccatcgacagactggccggcaagcccacacatgtgaatgtgtctgtggtcatggccgaggtggacggcacctgttattaa

SEQ ID NO: 7 - DNA, 711 bp, Artificial Sequence
  kappa light chain of conatumumab
atggcctgtcctggatttctgtgggccctcgtgatctctacctgcctggaattcagcatggccgagatcgtgctgacccagtctcctggcacactgtcactgtctccaggcgagagagctaccctgtcctgtagagcttcccagggcatctccagatcctacctggcctggtatcagcagaagcctggacaggctcccagcctgttgatctacggcgcttcttccagagccacaggcatccctgacagattctccggctctggctctggcaccgacttcaccctgaccatcagcagactggaacccgaggacttcgccgtgtactactgtcagcagttcggctcctctccttggacctttggccagggcaccaaggtggaaatcaagcggacagtggccgctccttccgtgttcatcttcccaccttccgacgagcagctgaagtccggcacagctagcgtggtctgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgttaa

SEQ ID NO: 8 - DNA, 564 bp, Artificial Sequence
  J chain precursor
atgaagaaccatttgcttttctggggagtcctggcggtttttattaaggctgttcatgtgaaagcccaagaagatgaaaggattgttcttgttgacaacaaatgtaagtgtgcccggattacttccaggatcatccgttcttccgaagatcctaatgaggacattgtggagagaaacatccgaattattgttcctctgaacaacagggagaatatctctgatcccacctcaccattgagaaccagatttgtgtaccatttgtctgacctctgtaaaaaatgtgatcctacagaagtggagctggataatcagatagttactgctacccagagcaatatctgtgatgaagacagtgctacagagacctgctacacttatgacagaaacaagtgctacacagctgtggtcccactcgtatatggtggtgagaccaaaatggtggaaacagccttaaccccagatgcctgctatcctgacctcgagtctagagggcccttcgaacaaaaactcatctcagaagaggatctgaatatgcataccggtcatcatcaccatcaccattga

(19) United States
(12) Patent Application Publication
(10) Pub. No.: US 2024/0382499 A1
(43) Pub. Date: Nov. 21, 2024

(54) INDUCERS OF SENESCENCE, IN COMBINATION WITH A SELECTIVE DEATH RECEPTOR 5 (DR5) AGONIST, FOR USE IN A METHOD OF TREATING CANCER

(71) Applicant: Stichting Het Nederlands Kanker Instituut-Antoni van Leeuwenhoek Ziekenhuis, Amsterdam (NL)

(72) Inventors: Rene BERNARDS, Amsterdam (NL); Liqin WANG, Amsterdam (NL)

(21) Appl. No.: 18/272,419

(22) PCT Filed: Jan. 17, 2022
(86) PCT No.: PCT/NL2022/050016

§ 371 Date: Jul. 14, 2023

(30) Foreign Application Priority Data
Jan. 15, 2021 (EP) 21151844.4

(51) Int. Cl. A61K 31/55; A61K 31/357; A61K 31/416; A61K 31/4545; A61K 31/517; A61K 31/519; A61K 31/704; A61K 39/00; A61K 45/06; A61P 35/00; C07K 16/28

(52) U.S. Cl. CPC A61K 31/55; A61K 31/357; A61K 31/416; A61K 31/4545; A61K 31/517; A61K 31/519; A61K 31/704; A61K 45/06; A61P 35/00; C07K 16/2878; A61K 2039/505

(57) ABSTRACT

The invention relates to an inducer of senescence, in combination with a short-lived, selective Death Receptor 5 (DR5) agonist, for use in a method of treating a patient suffering from a tumor. The invention further relates to a pharmaceutical composition, comprising a short-lived, selective Death Receptor 5 (DR5) agonist and, optionally, an inducer of senescence. The invention further relates to methods of treating a patient having a tumor, and to a selective, short-lived DR5 agonist, for use in a method of selectively killing senescent cells, including senescent cancer cells.

### FIELD

The invention relates to methods of treating cancer comprising an inducer of senescence and an agent that specifically kills senescent cells such as senescent cancer cells.

### 1. INTRODUCTION

Cancer remains difficult to treat, especially when disease is advanced. Combinations of different cancer drugs are used to suppress development of resistance, but such therapeutic approaches are often limited by toxicity. A radically different approach to cancer therapy was recently developed, which is not based on combinations of drugs, but rather on the sequential treatment with drugs, thereby avoiding drug combination toxicity (Wang et al., 2019. Nature 574:268-272). First, cells are induced to stop dividing and also acquire a major new vulnerability that is subsequently targeted by a second drug that selectively kills cells with the acquired vulnerability.

To accomplish this, advantage was taken of the notion that a cellular senescence response can be triggered in advanced cancers. Such senescent cancer cells have dramatic changes in gene expression and metabolism that might be exploited for their eradication. Validated functional genomics technology was used to identify genes whose suppression results in a senescence response in cancer cells (Wang et al., 2017. Cell Reports 21: 773-832). Using an animal model of liver cancer, proof of concept was delivered that induction of senescence, followed by treatment with an agent that specifically kills senescent cancer cells, resulted in dramatic responses (Wang et al., 2019. Nature 574: 268-272). This novel therapy is termed the “one-two punch” approach: the first therapy to induce senescence in cancer cells, the subsequent therapy to eradicate the senescent cells.

There is a need to identify triggers that induce senescence especially in advanced cancers, and to identify targets that are upregulated in advanced cancers upon induction of senescence and that can be used to specifically kill senescent cancer cells.

### BRIEF DESCRIPTION OF THE INVENTION

A CRISPR-based genetic screen was performed in cancer cells rendered senescent by multiple stimuli (alisertib, PLK4 inhibitors and etoposide) to identify vulnerabilities of senescent cells that are not shared by proliferating cancer cells. The results show that a selective Death Receptor 5 (DR5) agonist is able to selectively kill senescent cells, but not proliferating cells. The effects of a selective DR5 agonist on senescent cells could be markedly enhanced by co-provision of a Bromodomain Containing 2 (BRD2) inhibitor.

The invention therefore provides an inducer of senescence, in combination with a selective DR5 agonist, for use in a method of treating a patient suffering from a tumor. Said tumor optionally is not a melanoma.

Said selective DR5 agonist preferably has an in vivo half-life of 20 days or less,, such as more than 1 day, such as 2-20 days, 3-15 days, 3-6 days, or 4-9 days. A selective DR5 agonist with an in vivo half-life of less than 20 days may have similar anti-cancer effects as a longer lived DR5 agonist, but with reduced toxicity. Said selective Death Receptor 5 (DR5) agonist with a short half-life preferably is an antibody, preferably a human or humanized IgA or IgA-like antibody.

Said inducer of senescence preferably comprises, or is selected from, at least one of chemotherapy, ionizing radiation, a CDK4/6 inhibitor, a polo-like kinase 4 (PLK4) inhibitor, a topoisomerase II inhibitor, an aurora kinase B inhibitor. Said inducer of senescence preferably comprises, or is selected from, at least one of palbociclib, alisertib, PF-06873600, CFI-400945, etoposide, doxorubicin, and barasertib.

Said inducer of senescence and the selective DR5 agonist are preferably provided sequentially to the patient.

Said selective DR5 agonist optionally is combined with a Bromodomain Containing 2 (BRD2) inhibitor.

Said selective DR5 agonist preferably is an antibody, preferably a human or humanized antibody. More preferably, said selective DR5 agonist is a short-lived, human or humanized IgA or IgA-like antibody.

Said tumor preferably is a solid tumor such as lung cancer, breast cancer, colorectal cancer and/or liver cancer.

The invention further provides a selective DR5 agonist, wherein the selective DR5 agonist is an antibody, preferably a human or humanized IgA or IgA-like antibody, having an in vivo half-life of less than 20 days. Said selective DR5 agonist, preferably said short-lived, human or humanized IgA or IgA-like antibody is for use in a method of treating a patient suffering from a pathology involving senescent cells.

The invention further provides a pharmaceutical composition, comprising a short lived, selective DR5 agonist of the invention, optionally further comprising a BRD2 inhibitor.

The invention further provides a pharmaceutical composition, comprising an inducer of senescence and a selective DR5 agonist having an in vivo half-life of less than 20 days, optionally further comprising a BRD2 inhibitor. Said pharmaceutical preparation preferably is for use in a method of treating a patient suffering from a tumor.

The invention further provides a method of treating a patient having a tumor with a combination of an inducer of senescence and a selective DR5 agonist, comprising administering an inducer of senescence to said patient, followed by administering a selective DR5 agonist having an in vivo half-life of less than 20 days, optionally in combination with a BRD2 inhibitor. Said selective DR5 agonist, optionally in combination with a BRD2 inhibitor, preferably is provided at least 24 hours following the inducer of senescence. Said inducer of senescence, in combination with a selective Death Receptor 5 (DR5) agonist and, optionally, a BRD2 inhibitor, is preferably provided intermittently to the patient, for example every other day or every other week.

Said tumor preferably is a solid tumor such as lung cancer, breast cancer, colorectal cancer and/or liver cancer.

The invention further provides a selective DR5 agonist, preferably having an in vivo half-life of less than 20 days, such as 4-9 days, for use in a method of selectively killing of senescent cancer cells.

The invention further provides a method of treating a patient having a pathology involving senescent cells with a selective DR5 agonist having an in vivo half-life of less than 20 days, comprising administering the selective DR5 agonist of the invention, optionally in combination with a BRD2 inhibitor, to thereby treating said patient.

### FIGURE LEGENDS

FIG. 1: (A) gRNAs prioritized for further analysis were selected by the fold depletion of abundance in pre-and post-dox treatment in both alisertib induced senescent and proliferating cells (S2 and S1 samples compared to P2 and P1 samples), using methods as described previously (Evers et al., 2016. Nat Biotech 34: 631-633). (B) Western blot analysis of cFLIP in A549 cells stably infected with doxycycline-inducible Cas9 (iCas9) lentiviral vectors and two independent guide RNAs target cFLIP lentiviral vectors. Protein lysate were harvested after 4 days 1 μg/ml DOX treatment. Vinculin (VINC) served as loading control. (C) Cell viability was assessed by colony formation assay in the A549 cells stably infected with doxycycline-inducible Cas9 (iCas9) lentiviral vectors and two independent guide RNAs target cFLIP lentiviral vectors. The cells were firstly treated with 0.5 μM alisertib for 7 days to induce senescence. Afterwards, the senescent cells were suspended from alisertib then re-seeded in a 6-well plate and switched to 1 μg/ml DOX treatment for 10 days. Proliferating cells were taken as a control during doxycycline treatment. At the end of assay, cells were fixed, stained, and photographed after 10 days of culture. (D) Real-time PCR analysis of cFLIP in A549, Hep1 and HCT116 cells stably infected with two independent doxycycline-inducible shRNA-cFLIP lentiviral vector. mRNA was harvested after 4 days 1 μg/ml DOX treatment. GAPDH served as loading control. (E) Cell viability was assessed by colony formation assay in the A549, Hep1 and HCT116 cells stably infected with two independent doxycycline-inducible shRNA-cFLIP lentiviral. The cells were firstly treated with 0.5 μM alisertib for 7 days to induce senescence. Afterwards, the senescent cells were trypsinized and re-seeded in a 6-well plate and switched to 1 μg/ml DOX treatment for 10 days. Proliferating cells were taken as a control during doxycycline treatment. At the end of assay, cells were fixed, stained, and photographed after 10 days of culture. (F) Senescence associated beta-galactosidase (SA-β-gal) staining in A549 and Hep1 after treated with 0.5 μM alisertib, 1 μM Barasertib, 100 nM CFI-400945 and 2 μM Etoposide for one week. (G) Western blot analysis of cFLIP in A549 parental cells and two independent cFLIP knock out clones. Vinculin (VINC) served as loading control. (H) Cell viability was assessed by colony formation assay in the A549 parental and cFLIP null cells. The cells were treated with 0.5 μM alisertib, 1 μM Barasertib, 100 nM CFI-400945 and 2 μM Etoposide for one week. At the end of assay, cells were fixed, stained, and photographed. (I) Cell viability was assessed by colony formation assay in the Hep 1 parental and cFLIP null cells. The cells were treated with 0.5 μM alisertib, 1 μM Barasertib, 100 nM CFI-400945 and 2 μM Etoposide for one week. At the end of assay, cells were fixed, stained, and photographed.

FIG. 2: (A) Western blot analysis of DR4, DR5, cFLIP in A549 cells treated with different senescence inducers for 1 week. VINC served as loading control. (B) A549 cells were firstly treated with 0.5 μM alisertib for 7 days to induce senescence. Afterwards, the senescent cells were suspended from alisertib, re-seeded in a 96-well plate then switched to 200 ng/ml recombinant Lz-TRAIL. The growth curves were measured by Incucyte cell proliferation assay. (C) Alisertib induced senescent and proliferating A549, H2030, H358, HCT116, MDA-MB-231 and MCF-7 cells were seeded into a 384-well plate and treated with Lz-TRAIL for 120 hrs. Cell viability were measured using CellTiter-Blue to generate dose response curve. (D) Senescence associated beta-galactosidase (SA-β-gal) staining in a cell line panel after treated with 0.5 μM alisertib for one week. (E) Cell viability 20 was assessed by colony formation assay in the cell line panel. The cells were firstly treated with 0.5 μM alisertib for 7 days to induce senescence. Afterwards, the senescent cells were suspended from alisertib then re-seeded in a 12-well plate and switched to Lz-TRAIL and ABT263/Navitoclax treatment for 120 days. Proliferating cells were taken as a control. At the end of assay, cells were fixed, stained, and photographed after 10 days of culture.

FIG. 3: (A) Human NF-kB pathway protein array analysis on the senescent A549 cells generated with 0.5 μM alisertib for 7 days. Proliferating cells were used as a control. (B) Real-time PCR analysis of DR4 and DR5 in A549 cells stably infected with lentiviral shRNAs against DR4 or DR5. GAPDH served as control. (C) Cell viability was assessed by colony formation assay in the A549 cells lentiviral shRNAs against DR4 or DR5. The cells were firstly treated with 0.5 μM alisertib for 7 days to induce senescence. Afterwards, the senescent cells were trypsinized and re-seeded in a 6-well plate and switched to 200 ng/ml Lz-TRAIL for 120 hours. Proliferating cells were taken as a control. At the end of assay, cells were fixed, stained, and photographed after 10 days of culture. (D-F) Alisertib induced senescent and proliferating cell line panel were seeded into a 384-well plate and treated with conatumumab or with navitoclax for 120 hrs. Cell viability were measured using CellTiter-Blue to generate dose response curve.

FIG. 4: (A) Tumor growth of Hep1 parental cells in the flanks of Balb/c nude mice subcutaneously injected with 1*10⁷ cells. When tumors reached approximately 200 mm³, assigned to either control, 30 mg/kg alisertib (oral gavage), 5 μg conatumumab (intraperitoneal injection) and the combination. The drugs were administrated during week days and suspended during the weekend. (B) Tumor growth of A549 parental cells in the flanks of Balb/c nude mice subcutaneously injected with 5*10⁶ cells. When tumors reached approximately 200 mm3, assigned to either control, 30 mg/kg alisertib (oral gavage), 10 μg conatumumab (intraperitoneal injection) and the combination. The drugs were administrated during week days and suspended during the weekend. (C-E) Cell viability was assessed by colony formation assay in the A549 and Hep1 cells. The cells were firstly treated with different senescence inducers for 7 days to induce senescence. Afterwards, the senescent cells were suspended from the senescence inducers then re-seeded in a 12-well plate and switched to conatumumab or Lz-TRAIL treatment for 5 days. Proliferating cells were taken as a control. At the end of assay, cells were fixed, stained, and photographed.

FIG. 5: (A) gRNAs prioritized for further analysis were selected by the fold depletion of abundance in pre-and post-dox treatment in both Etoposide or CFI-400945 induced senescent and proliferating A549 lung cancer cells (S2 and S1 samples compared to P2 and P1 samples) and shown with the MA plots, using methods as described previously. (B) gRNAs prioritized for further analysis were selected by the fold depletion of abundance in pre-and post-dox treatment in alisertib induced senescent Hep1 liver cancer cells (S2 compared to S1 samples) and shown with the volcano plots.

FIG. 6: (A) Dose-response curves determined by cell viability using cell titer blue for NEO2734 in the presence of 0.5 microgram/ml conatumumab. Shown are dose response curves for PC9, TFK, EGI and Hep 1 cancer cells, and BJ and Rpe1 primary cells. (B) RNA sequencing results.

FIG. 7: (A) Cell viability for the BRD2 inhibitor iBET in the presence of 0.5 microgram/ml conatumumab. (B) Combined BRD2 inhibition and DR5 activation improves killing of senescent cancer cells, but not of proliferating cancer cells.

FIG. 8: Cell viability was assessed by colony formation assay using low doses of NEO2734 and conatumab. (A) Cell viability was assessed by a colony formation assay using low dose 0.25 μM of NEO2734 and 0.125 μg/ml conatumumab on Hep1 cells made senescent by one-week treatment of 0.5 μM alisertib, 1 μM barasertib, 50 nM CFI-400945 or 2 μM Etoposide. (B) Colony formation using low dose 0.25 μM of NEO2734 and 0.125 μg/ml conatumumab on Hep1, 0.25 μM of NEO2734 and 1 μg/ml conatumumab on A549 cells made senescent by one-week treatment of different senescence inducers (0.5 μM PF-06873600, 100 nM doxorubicin, 1 μM TAS-119 or combination of 5 nM trametinib plus 0.5 μM palbociclib.

FIG. 9: Examination of NEO2734 plus conatumumab senolytic cocktail efficacy on bleomycin-induced senescent A549 cells as an idiopathic pulmonary fibrosis model. (A) Western blot analysis on bleomycin treated A549 cells. (B) Senescence associated beta-galactosidase staining on one week of bleomycin-induced senescent A549 cells. (C) Colony formation on bleomycin-induced senescent A549 cells treated with 0.25 μM NEO2734 plus 2 μg/ml conatumumab (see Hui et al., 2005. Chest 128: 2247-2261).

FIG. 10: Colony formation in proliferating and alisertib-induced senescent A549 (A), Hep1 (B) and MM231 (C) cells with IgG1-cona or IgA-cona-dim DR5 agonist antibodies. Colony formation in proliferating and alisertib-induced senescent A549 cells treated with the low dose of 0.125 μg/ml IgA-cona-dim, 0.25 μM NEO2734 and 10 μM Z-VAD-FMK (D). Colony formation using low dose 0.25 μM of NEO2734 and 0.125 μg/ml IgA-cona-dim A549 cells made senescent by one-week treatment with 1 μM barasertib, 0.5 μM PF-06873600, 100 nM doxorubicin, 50 nM CFI-400945, 2 μM Etoposide or 1 μM TAS-119 (E).

### DETAILED DESCRIPTION OF THE INVENTION

### DEFINITIONS

The term “senescence”, as is used herein, refers to a state of a cell that is characterized by having an essentially permanent growth arrest in the G1 or G2/M phase of the cell cycle. A senescent cell is essentially irresponsive to proliferation-cues. The term “senescent cell”, as used herein, includes a cell that is characterized by (1) an essentially permanent growth arrest; (2) loss of proliferation markers such as cyclin A, MCM-3 and/or PCNA; (3) insensitivity to growth cues; (4) induction of a senescence-associated β-galactosidase (SA-B-Gal); and (5) nuclear export of alarmin, a High Mobility Group Box 1 protein. The phenomenon of senescence can occur at the end of the proliferative lifespan of normal cells or in normal or tumor cells in response to, for example, chemotherapeutic agents, radiation, or other cellular insults. Senescent cells often remain metabolically active and commonly adopt an immunogenic phenotype consisting of a pro-inflammatory secretome. A senescent cell often has upregulated expression of a cell cycle inhibitor like p16/p21.

The term “pathology involving senescent cells”, as is used herein, refers to pathologies such as osteoporosis, frailty, cardiovascular diseases, osteoarthritis, pulmonary fibrosis, renal diseases, neurodegenerative diseases, hepatic steatosis, metabolic dysfunction, and senescent fibroblast-mediated pathologies such as idiopathic pulmonary fibrosis. A pathology involving senescent cells may also be referred to as an age-related disease.

The term “inducer of senescence”, as is used herein, refers to the induction of a cellular stress response that results in an essentially permanent growth arrest of the cell. Senescence can be triggered by a diverse set of signals, including shortening of telomeres, DNA damage, activation of oncogenes, and oxidative stress. In the context of this invention, an inducer or senescence is preferably selected from a chemotherapeutic agent, ionizing radiation, a CDK4/6 inhibitor, a Polo-like kinase 4 (PLK4) inhibitor, a Topoisomerase II inhibitor, and an Aurora kinase inhibitor, preferably an Aurora kinase B inhibitor.

The term “cyclin-dependent kinase 4/6 (CDK4/6)”, as is used herein, refers to two closely related members of a family of serine/threonine protein kinases that participate in cell cycle regulation, CDK4 and CDK6. Both members are cyclin D-dependent kinases that regulate entry into the DNA synthetic(S) phase of the cell-division cycle in a retinoblastoma protein-dependent manner.

The term “inhibitor of cyclin-dependent kinase 4/6”, as is used herein, refers to a molecule that inhibits CDK4/6. A preferred CDK4/6 inhibitor is selective for CDK4/6, when compared to other serine/threonine protein kinases such as CDK1 and CDK2, meaning that the molecule is at least two times more potent, preferably at least five times more potent, in inhibiting CDK4/6, when compared to other serine/threonine protein kinases.

The term “polo-like kinase 4 (PLK4)”, as is used herein, refers to a serine/threonine protein kinase that plays a central role in centriole duplication. The human gene encoding PLK4 resides on chromosome 4q28.1, and is characterized by HGNC entry code 11397; Entrez Gene entry code 10733; and Ensembl entry code ENSG00000142731. The PLK4 protein is characterized by UniProt entry code O00444.

The term “PLK4 inhibitor”, as is used herein, refers to a molecule that inhibits PLK4. A preferred PLK4 inhibitor is selective for PLK4, when compared to other polo-like serine/threonine protein kinases such as PLK1, PLK2 and PLK3, meaning that the molecule is at least two times more potent, preferably at least five times more potent, in inhibiting PLK4, when compared to other serine/threonine protein kinases such as other polo-like serine/threonine protein kinases.

The term “topoisomerase II”, as is used herein, refers to a DNA Type IIA topoisomerase that is involved in the separation of chromosomal daughter strands during replication. Failure to separate these strands leads to cell death. The human gene encoding topoisomerase II resides on chromosome 17q21.2, and is characterized by HGNC entry code 11989; Entrez Gene entry code 7153; and Ensembl entry code ENSG00000131747. The topoisomerase II protein is characterized by UniProt entry code P11388.

The term “topoisomerase II inhibitor”, as is used herein, refers to a molecule that inhibits topoisomerase II. A preferred topoisomerase II inhibitor is selective for topoisomerase II, when compared to other topoisomerases such as topoisomerase I and topoisomerase III, meaning that the molecule is at least two times more potent, preferably at least five times more potent, in inhibiting topoisomerase II, when compared to other topoisomerases such as topoisomerase I and topoisomerase III.

The term “aurora kinase B”, as is used herein, refers to serine/threonine protein kinase that is a component of the chromosomal passenger complex that acts as a key regulator of mitosis. The human gene encoding aurora kinase B resides on chromosome 17p13.1, and is characterized by HGNC entry code 11390; Entrez Gene entry code 9212; and Ensembl entry code ENSG00000178999. The aurora kinase B protein is characterized by UniProt entry Q96GD4.

The term “aurora kinase B inhibitor”, as is used herein, refers to a molecule that inhibits aurora kinase B. A preferred aurora kinase B inhibitor is selective for aurora kinase B, when compared to other aurora kinases such as aurora kinase A and aurora kinase C, meaning that the molecule is at least two times more potent, preferably at least five times more potent, in inhibiting aurora kinase B, when compared to other aurora kinases such as aurora kinase A and aurora kinase C.

The term “Death Receptor 5 or DR5”, as is used herein, refers to protein member 10b of the tumor necrosis factor (TNF) Receptor Superfamily. Alternative names are TRAILR2 and TRICK2. The human gene encoding DR5 resides on chromosome 8p21.3, and is characterized by HGNC entry code 11905; Entrez Gene entry code 8795; and Ensembl entry code ENSG00000120889. The DR5 protein is characterized by UniProt entry code O14763.

The term “DR5 agonist”, as is used herein, refers to a molecule such as an antibody that binds and activates DR5. DR5 harbors a death domain, a stretch of about 90 amino acid residues that is required and sufficient to activate the apoptotic machinery. Binding and activation of DR5 by a DR5 agonist thus results in the induction of apoptosis.

The term “selective DR5 agonist”, as is used herein, refers to a molecule that binds and activates specifically DR5. Binding of a selective DR5 agonist to DR5 is at least two times more potent, preferably at least five times more potent, in activating DR5, when compared to other death receptor proteins such as DR4.

The term “Bromodomain Containing 2 (BRD2)”, as is used herein, refers to a transcriptional regulator that belongs to the BET (bromodomains and extra terminal domain) family of proteins. The human gene encoding BRD2 maps to chromosome 6p21.3, and is characterized by HGNC entry code 1103; Entrez Gene entry code 6046; and Ensembl entry code ENSG00000204256. The BRD2 protein is characterized by UniProt entry code P25440.

The term “BRD2 inhibitor”, as is used herein, refers to a molecule that binds and inhibits BRD2. A preferred BRD2 inhibitor is selective for BRD2, when compared to other BET domain proteins such as BRD3, BRD4, and BRDT, meaning that the molecule is at least two times more potent, preferably at least five times more potent, in inhibiting BRD2, when compared to other BET proteins such as BRD3, BRD4, and BRDT. A further preferred BRD2 inhibitor is selective for a first BET domain in BRD2, when compared to a second BET domain in BRD2. meaning that the molecule is at least two times more potent, preferably at least five times more potent, in inhibiting a first BET domain, when compared to a second BET domain in BRD2. A further preferred BRD2 inhibitor is selective for a second BET domain in BRD2, when compared to a first BET domain in BRD2. meaning that the molecule is at least two times more potent, preferably at least five times more potent, in inhibiting a second BET domain, when compared to a first BET domain in BRD2.

The term “peptide”, as used herein, refers to a molecule with an amino acid chain of between 5 and 100 amino acid residues, preferably between 10 and 50 amino acid residues. The term peptide includes a peptide in which one or more of the amino acid monomers have been modified, for example by acetylation, amidation and/or glycosylation.

The term “peptide analogue”, as used herein, refers to peptidomimetics which are or which comprise small peptide-like chains such as peptoids and β-peptides designed to mimic a peptide. The altered chemical structure is preferably designed to adjust one or more properties such as, for example, stability, of a peptide. cell-penetrating domain.

The term “combination”, as is used herein, refers to the administration of effective amounts of an inducer of senescence and a selective DR5 agonist to a patient in need thereof. Said inducer of senescence and a selective DR5 agonist may be provided in one pharmaceutical preparation, or as two distinct pharmaceutical preparations.

The term “antibody”, as is used herein, includes reference to classical heterodimers of heavy and light chain antibodies, single heavy chain variable domain antibody such as a camelid VHH, a shark immunoglobulin-derived variable new antigen receptor, and scFv, tandem scFv, scFab, and improved scFab (Koerber et al., 2015. J Mol Biol 427: 576-86). The heavy and light chains of classical antibodies comprise a variable region (V region) and a constant or C region. As described herein, the amino acid sequence and structure of the variable region of heavy and light chains of classical antibodies is comprised of four framework regions or ‘FR’, which are referred to herein as ‘Framework region 1’ or ‘FRl’; as ‘Framework region 2’ or ‘FR2’; as ‘Framework region 3’ or ‘FR3’; and as ‘Framework region 4’ or ‘FR4’, respectively; which framework regions are interrupted by three complementary determining regions or ‘CDR's’, which are referred to herein as ‘Complementarity Determining Region l’ or ‘CDR1’; as ‘Complementarity Determining Region 2’ or ‘CDR2’; and as ‘Complementarity Determining Region 3’ or ‘CDR3’, respectively. The term antibody also includes an antibody-like molecule that is not structurally related to an antibody. Such antibody-like molecules include, for example, a designed ankyrin repeat protein, a binding protein that is based on a Z domain of protein A, a binding protein that is based on a fibronectin type III domain, engineered lipocalin, and a binding protein that is based on a human Fyn SH3 domain (Skerra, 2007. Current Opinion Biotechnol 18: 295-304; Škrlec et al., 2015. Trends Biotechnol 33: 408-418).

The term “anti-DR5 IgA or IgA-like antibody”, as is used herein, refers to any and all anti-DR5 antibodies that comprise at least part of a CD89-interacting domain in the Cα3 domain, including at least amino acid residues L441A442 (Pleass et al., 1999. JBC 274: 23508-23514). The inclusion of an IgA-derived CD89-interacting domain with at least amino acid residues L441A442 will contribute to a shortened in vivo half-life of the anti-DR5 IgA or IgA-like antibody of less than 20 days, such as 3-9 days.

The term “selective binding”, or grammatical variations thereof, as used herein, refers to the number of different types of antigens or their epitopes to which a particular antibody can bind. The specificity of an antibody can be determined based on affinity. A specific antibody preferably has a binding affinity Kd for its epitope of less than 10⁻⁷ M, preferably less than 10⁻⁸ M, most preferable less than 10⁻⁹ M.

The term “format”, or “antibody format’, as is used herein, refers to the class or isotype of an antibody, which for human antibodies is selected from immunoglobulins (Ig) IgA, IgD, IgE, IgG, or IgM, which are in part determined by the constant region. The constant region includes sites involved in interactions with other components of the immune system.

The term “reformatting”, as is used herein, refers to the grafting of CDRs of one format of antibody to another format. For example, CDRs from an IgE antibody may be grafted to the frame work regions of an IgG antibody.

### 4.2 Methods of the Invention

The invention provides an use of an inducer of senescence, in combination with a selective Death Receptor 5 (DR5) agonist, in the preparation of a medicament for treating a patient suffering from a tumor. Said combination preferably includes firstly providing said patient with the inducer of senescence, followed by the provision of the selective DR5 agonist. Said selective DR5 agonist is preferably provided at least 24 hours, preferably at least 3 days such as at least 4 days, at least 5 days, at least 6 days, at least 7 days, following the inducer of senescence. Said selective DR5 agonist is preferably provided at most 14 days, such as at most 10 days, following the inducer of senescence. Said an inducer of senescence and said selective DR5 agonist are preferably provided intermittently to the patient, for example every other week, biweekly, or once a month.

The invention provides a method of treating a patient having a tumor with a combination of an inducer of senescence and a selective DR5 agonist. Said method preferably comprises firstly providing said patient with the inducer of senescence, followed by the provision of the selective DR5 agonist. Said selective DR5 agonist is preferably provided at least 24 hours, preferably at least 3 days such as at least 4 days, at least 5 days, at least 6 days, at least 7 days, following the inducer of senescence. Said selective DR5 agonist is preferably provided at most 14 days, such as at most 10 days, following the inducer of senescence. Said inducer of senescence and said selective DR5 agonist are preferably provided intermittently to the patient, for example every other week, biweekly, or once a month.

The invention provides a combination of an inducer of senescence and a selective DR5 agonist for use in a method of treating a patient having a tumor. Said combination preferably comprises firstly providing said patient with the inducer of senescence, followed by the provision of the selective DR5 agonist. Said selective DR5 agonist is preferably provided at least at least 24 hours, preferably at least 3 days such as at least 4 days, at least 5 days, at least 6 days, at least 7 days, following the inducer of senescence. Said selective DR5 agonist is preferably provided at most 14 days, such as at most 10 days, following the inducer of senescence. Said an inducer of senescence and said selective DR5 agonist are preferably provided intermittently to the patient, for example every other week, biweekly, once a month.

Said tumor especially is a malignant neoplasm, and may include a blood tumor such as a leukemia, a lymphoma, and a myeloma; a tumor of mesenchymal origin such as a sarcoma; and a tumor of epithelial origin.

Said tumor preferably is a solid tumor, including a germ cell tumor such as a teratoma, a yolk sac tumor, a choriocarcinoma, an embryonal carcinoma, a seminoma, or a mixed germ cell tumor such as a teratocarcinoma; a Wilms' tumor, a mesothelioma, a melanoma, a sarcoma and a carcinoma. Said carcinoma includes adenoid cystic carcinoma, bladder carcinoma, breast cancer, cervical cancer, colorectal cancer, ductal carcinoma, endometrial cancer, esophageal cancer, gastric cancer, kidney cancer, laryngeal cancer, liver cancer, lung cancer, including small cell and non-small cell lung cancer, nasopharyngeal cancer, oral cancer, ovarian cancer, pancreatic cancer, penile cancer, peritoneal cancer, prostate cancer, renal cell carcinoma, thyroid cancer, and a vaginal cancer, preferably a carcinoma such as a lung cancer, a breast cancer, a colorectal cancer and/or a liver cancer.

Said tumor optionally is not a melanoma.

Said inducer of senescence preferably comprises at least one of a chemotherapeutic agent, ionizing radiation, a CDK4/6 inhibitor, a polo-like kinase 4 (PLK4) inhibitor, a topoisomerase II inhibitor, an aurora kinase B inhibitor.

Said chemotherapeutic agent preferably is selected from an alkylating agent such as nitrogen mustard, e.g. cyclophosphamide, mechlorethamine or mustine, uramustine and/or uracil mustard, melphalan, chlorambucil, ifosfamide; a nitrosourea compound such as carmustine, lomustine, and streptozocin; an alkyl sulfonate such as busulfan; an ethylenime such as thiotepa and analogues thereof; a hydrazine/triazine such as dacarbazine, altretamine, mitozolomide, temozolomide, altretamine, procarbazine, and temozolomide; an intercalating agent such as a platinum-based compound like cisplatin, carboplatin, nedaplatin, oxaliplatin and satraplatin; an anthracycline such as doxorubicin, daunorubicin, epirubicin and idarubicin; a folate targeting agent such as methotrexate, 5-fluorouracil, folinic acid, and capecitabine, a tubulin targeting agent such as vinorelbine, vinblastine, vincristine, and docetaxel; mitomycin-C, dactinomycin, bleomycin, adriamycin, and mithramycin.

Said ionizing radiation preferably is selected from high-energy particles or waves, such as x-rays, gamma rays, electron beams, or protons, to destroy or damage cancer cells. Radiation therapy works by introducing breaks in the DNA of a tumor cell, thereby preventing said tumor cell growth.

Said CDK4/6 inhibitor preferably is selected from palbociclib (571190-30-2; PD0332991; 6-acetyl-8-cyclopentyl-5-methyl-2-[(5-piperazin-1-ylpyridin-2-yl)amino]pyrido[2,3-d]pyrimidin-7-one), ribociclib (LEE011; 7-cyclopentyl-N,N-dimethyl-2-[(5-piperazin-1-ylpyridin-2-yl)amino]pyrrolo[2,3-d]pyrimidine-6-carboxamide)pyrimidine-6-carboxamide), abemaciclib (LY2835219; N-[5-[(4-ethylpiperazin-1-yl)methyl]pyridin-2-yl]-5-fluoro-4-(7-fluoro-2-methyl-3-propan-2-ylbenzimidazol-5-yl)pyrimidin-2-amine), trilaciclib (4-[[5-(4-methylpiperazin-1-yl)pyridin-2-yl]amino]spiro[1,3,5,11-tetrazatricyclo[7.4.0.02,7]trideca-2,4,6,8-tetraene-13,1′-cyclohexane]-10-one), lerociclib (formerly referred to as G1T38; 2′-((5-(4-isopropylpiperazin-1-yl)pyridin-2-yl)amino)-7′,8′-dihydro-6′H-spiro[cyclohexane-1,9′-pyrazino[1′,2′: 1,5]pyrrolo[2,3-d]pyrimidin]-6′-one), and PF-06873600 (6-(difluoromethyl)-8-[(1R,2R)-2-hydroxy-2-methylcyclopentyl]-2-[(1-methylsulfonylpiperidin-4-yl)amino]pyrido[2,3-d]pyrimidin-7-one).

Said polo-like kinase 4 (PLK4) inhibitor preferably is selected from R1530 (5-(2-chlorophenyl)-7-fluoro-8-methoxy-3-methyl-2,10-dihydrobenzo[e]pyrazolo[4,3-b][1,4]diazepine), CFI-400945 ((2′S,3R)-2′-[3-[(E)-2-[4-[[(2S,6R)-2,6-dimethylmorpholin-4-yl]methyl]phenyl]ethenyl]-1H-indazol-6-yl]-5-methoxyspiro[1H-indole-3,1′-cyclopropane]-2-one), centrinone (2-[2-fluoro-4-[(2-fluoro-3-nitrophenyl)methylsulfonyl]phenyl]sulfanyl-5-methoxy-N-(5-methyl-1H-pyrazol-3-yl)-6-morpholin-4-ylpyrimidin-4-amine), centrinone B (LCR-323), KW-2449 ([4-[(E)-2-(1H-indazol-3-yl)ethenyl]phenyl]-piperazin-1-ylmethanone), and axitinib (N-methyl-2-[[3-[(E)-2-pyridin-2-ylethenyl]-1H-indazol-6-yl]sulfanyl]benzamide).

Said topoisomerase II inhibitor preferably is selected from a podophyllotoxin derivative such as etoposide ((5S,5aR,8aR,9R)-5-[[(2R,4aR,6R,7R,8R,8aS)-7,8-dihydroxy-2-methyl-4,4a,6,7,8,8a-hexahydropyrano[3,2-d][1,3]dioxin-6-yl]oxy]-9-(4-hydroxy-3,5-dimethoxyphenyl)-5a,6,8a,9-tetrahydro-5H-[2]benzofuro[6,5-f][1,3]benzodioxol-8-one), etoposide phosphate ([4-[(5S,5aR,8aR,9R)-5-[[(2R,4aR,6R,7R,8R,8aS)-7,8-dihydroxy-2-methyl-4,4a,6,7,8,8a-hexahydropyrano[3,2-d][1,3]dioxin-6-yl]oxy]-8-oxo-5a,6,8a,9-tetrahydro-5H-[2]benzofuro[5,6-f][1,3]benzodioxol-9-yl]-2,6-dimethoxyphenyl] dihydrogen phosphate), and teniposide (5S,5aR,8aR,9R)-9-(4-hydroxy-3,5-dimethoxyphenyl)-8-oxo-5,5a,6,8,8a,9-hexahydrofuro[3′,4′: 6,7]naphtho[2,3-d][1,3]dioxol-5-yl 4,6-O-(2-thienylmethylene)-β-D-glucopyranoside); ICRF-193 (4-[(2R,3S)-3-(3,5-dioxo-1-piperazinyl)-2-butanyl]-2,6-piperazinedione); genistein (5,7-dihydroxy-3-(4-hydroxyphenyl)chromen-4-one); amsacrine (N-[4-(acridin-9-ylamino)-3-methoxyphenyl]methanesulfonamide); mitoxantrone (1,4-dihydroxy-5,8-bis[2-(2-hydroxyethylamino)ethylamino]anthracene-9,10-dione; dihydrochloride); resveratrol (5-[(E)-2-(4-hydroxyphenyl)ethenyl]benzene-1,3-diol); and HU-331 ((1′R,6′R)-6-hydroxy-6′-isopropenyl-3′-methyl-4-pentyl-1,1′-bi(cyclohexane)-2′,3,6-triene-2,5-dione).

A person skilled in the art will appreciate that an anthracycline such as doxorubicin, daunorubicin, epirubicin and idarubicin, which are listed herein above as a chemotherapeutic agent, can also be used a topoisomerase II inhibitor.

Said aurora kinase inhibitor preferably is selected from a alisertib (MLN8237; 4-[[9-chloro-7-(2-fluoro-6-methoxyphenyl)-5H-pyrimido[5,4-d][2]benzazepin-2-yl]amino]-2-methoxybenzoic acid), AMG 900 (N-[4-[3-(2-aminopyrimidin-4-yl)pyridin-2-yl]oxyphenyl]-4-(4-methylthiophen-2-yl)phthalazin-1-amine). AT9283 (1-cyclopropyl-3-[5-[6-(morpholin-4-ylmethyl)-1H-benzimidazol-2-yl]-1H-pyrazol-4-yl]urea; hydrochloride), barasertib (AZD1152-HQPA; 2-[ethyl-[3-[4-[[5-[2-(3-fluoroanilino)-2-oxoethyl]-1H-pyrazol-3-yl]amino]quinazolin-7-yl]oxypropyl]amino]ethyl dihydrogen phosphate), CCT137690 (3-[[4-[6-bromo-2-[4-(4-methylpiperazin-1-yl)phenyl]-1H-imidazo[4,5-b]pyridin-7-yl]piperazin-1-yl]methyl]-5-methyl-1,2-oxazole), CYC116 (4-methyl-5-(2-(4-morpholinophenylamino)pyrimidin-4-yl)thiazol-2-amine), ENMD-2076 ((2S,3S)-2,3-dihydroxybutanedioic acid; 6-(4-methylpiperazin-1-yl)-N-(5-methyl-1H-pyrazol-3-yl)-2-[(E)-2-phenylethenyl]pyrimidin-4-amine), GSK1070916 (3-[4-[4-[2-[3-[(dimethylamino)methyl]phenyl]-1H-pyrrolo[2,3-b]pyridin-4-yl]-1-ethylpyrazol-3-yl]phenyl]-1,1-dimethylurea), hesperadin (N-[2-hydroxy-3-[C-phenyl-N-[4-(piperidin-1-ylmethyl)phenyl]carbonimidoyl]-1H-indol-5-yl]ethanesulfonamide), MK-5108 (VX-689; 4-(3-chloro-2-fluorophenoxy)-1-[[6-(1,3-thiazol-2-ylamino)pyridin-2-yl]methyl]cyclohexane-1-carboxylic acid). MK-8745 ((3-chloro-2-fluorophenyl)-[4-[[6-(1,3-thiazol-2-ylamino)pyridin-2-yl]methyl]piperazin-1-yl]methanone), MLN8054 (4-((9-chloro-7-(2,6-difluorophenyl)-5H-benzo[c]pyrimido[4,5-e]azepin-2-yl amino)benzoic acid), PF-03814735 (N-{2-[(1R,8S)-4-{[4-(Cyclobutylamino)-5-(trifluoromethyl)-2-pyrimidinyl]amino}-11-azatricyclo[6.2.1.02,7]undeca-2,4,6-trien-11-yl]-2-oxoethyl}acetamide. reversine (6-N-cyclohexyl-2-N-(4-morpholin-4-ylphenyl)-7H-purine-2,6-diamine), TAK-901 (5-(3-ethylsulfonylphenyl)-3,8-dimethyl-N-(1-methylpiperidin-4-yl)-9H-pyrido[2,3-b]indole-7-carboxamide), VX-680 (MK-0457, tozasertib) N-[4-[4-(4-Methylpiperazin-1-yl)-6-[(5-methyl-1H-pyrazol-3-yl)amino]pyrimidin-2-yl]sulfanylphenyl]cyclopropanecarboxamide, ZM-447439 (N-[4-[[6-methoxy-7-(3-morpholin-4-ylpropoxy)quinazolin-4-yl]amino]phenyl]benzamide) and TAS-119 (1-(2,3-dichlorobenzoyl)-4-[[5-fluoro-6-[(5-methyl-1H-pyrazol-3-yl)amino]pyridin-2-yl]methyl]piperidine-4-carboxylic acid).

A preferred inducer of senescence comprises at least one of palbociclib, alisertib, CFI-400945, etoposide, doxorubicin, and barasertib.

Said inducer of senescence is combined with a selective DR5 agonist. Said inducer of senescence may be administrated separately from, or sequentially to the DR5 agonist. When administered as two distinct pharmaceutical preparations, they are preferably administered on different days to a patient in need thereof, and using a similar or dissimilar administration protocol, e.g. daily, twice daily, biweekly, orally and/or by infusion.

Said combination of an inducer of senescence and a selective DR5 agonist is preferably administered repeatedly according to a protocol that depends on the patient to be treated (age, weight, treatment history, etc.), which can be determined by a skilled physician. Said treatment protocol may include administration of an inducer of senescence in a first time span, followed by administration of a selective DR5 agonist in a second time span.

For example, said treatment protocol may include daily administration of an inducer of senescence in week 1, followed by daily administration of a selective DR5 agonist in week 2. Said treatment protocol may also include bi-daily administration of an inducer of senescence in weeks 1 and 2, followed by daily or bi-daily administration of a selective DR5 agonist in weeks 3 and 4.

A person skilled in the art will appreciate that the length of administration of an inducer of senescence may be dependent on the type of tumor, whereby a specific tumor type may require more time for induction of senescence, when compared to another tumor type.

Said combination of an inducer of senescence and a selective DR5 agonist is preferably administered intermittently according to a protocol that depends on the patient to be treated (age, weight, treatment history, etc.), which can be determined by a skilled physician. Said treatment protocol may include sequential administration of an inducer of senescence and a selective DR5 agonist every 2 days, every 3 days, every 5 days, every 10 days, every 21 days, every 28 days, or even every 2 months. A period of administration of an inducer of senescence and a selective DR5 agonist may be followed by a period of 1-28 days, such as 7 days or 14 days, in which no combination of an inducer of senescence and a selective DR5 agonist are administered.

In addition, cellular senescence may be considered an age-related disease, which also may play a role in certain pathologies such as osteoporosis, frailty, cardiovascular diseases, osteoarthritis, pulmonary fibrosis, renal diseases, neurodegenerative diseases, hepatic steatosis, metabolic dysfunction, and senescent fibroblast-mediated pathologies such as idiopathic pulmonary fibrosis. Therapeutic strategies that safely interfere with cellular senescence, such as the selective elimination of senescent cells, are gaining attention, with several programs now in clinical studies. For example, the threat of COVID-19 is not only the pneumonia resulting from the infection, but also the following long-term health effect, indicating the seriousness of the disease. More than 20% of SARS survivors developed pulmonary fibrosis within a year, as a long-term damage from the infection (Hui et al., 2005. Chest 128: 2247-2261; Xie et al., 2005. Respir Res 6: 5). In COVID-19 patients, it was also reported that symptoms of fibrosis were present (Ye et al., 2020. Eur Radiol 30: 4381-4389; Bazdyrev et al., 2021. Pharmaceuticals 14(8): 807), and more data supporting the prevalence of post-COVID-19 pathologies is being released as the pandemic continues.

The invention therefore provides a selective DR5 agonist, for use in a method of treating a patient suffering from a pathology involving senescent cells. Said selective DR5 agonist preferably has an in vivo half-life of 20 days or less, such as 4-9 days, preferably 3-6 days. Said selective DR5 agonist optionally is combined with a Bromodomain Containing 2 (BRD2) inhibitor. Said selective DR5 agonist preferably is an antibody, preferably a human or humanized antibody, preferably an human or humanized IgA or IgA-like antibody.

As is shown in the examples, an anti-DR5 IgA or IgA-like antibody according to the invention, and especially a dimerized anti-DR5 IgA or IgA-like antibody, was found to be a more potent senolytic inducer than a related anti-DR5 IgG antibody. In addition, the enhanced potency allows an anti-DR5 IgA or IgA-like antibody to be used at a lower dosage, thereby even further reducing potential side effects and toxicity, in addition to the reduced half-life, when compared to a conventional anti-DR5 antibody such as an anti-DR5 IgG antibody.

In addition, the presence of a CD89 interacting domain on an anti-DR5 IgA or IgA-like antibody according to the invention may result in the recruitment of CD89-expressing innate immune cells, such as neutrophils, to the DR5-expressing senescent cells, thereby mediating antibody-dependent cellular cytotoxicity (ADCC) of the DR5-expressing senescent cells. In addition, human neutrophils have been reported to release catalytically active neutrophil elastase (ELANE) to kill many cancer cell types, while sparing non-cancer cells (Cui et al., 2021. Cell 184: 3163-3177).

Said selective DR5 agonist preferably has an in vivo or biological half-life of less than 20 days, such as 19 days, 18 days, 17 days, 16 days, 15 days, 14 days, 13 days, 12 days, 11 days, 10 days, 9 days, 8 days, 7 days, 6 days, 5 days, 4 days, 3 days, 2 days, 1 day, or less than 1 day such as 0.5 days. A biological half-life is the time it takes for said selective DR5 agonist to reach 50% of the initial concentration in blood plasma.

Factors that may influence said half-life are breakdown of the agonist, and/or clearance by liver or kidney. Further relevant factors include accumulation in tissues and interaction with other receptors.

Factors that may prolong half-life of a selective DR5 agonist include binding to a serum protein such as serum albumin, lipidation, and pegylation, as is known to a person skilled in the art. Factors that may reduce half-life of a selective DR5 agonist include the generation of non-natural molecules, such as genetically engineered antibodies.

As is indicated herein above, said selective DR5 agonist is specific for DR5, meaning that the concentration at which said selective DR5 agonist binds to and activates DR5 is at least two times lower, when compared to the concentration at which said selective DR5 agonist binds to and activates DR4, preferably at least five times lower. A selective DR5 agonist with an in vivo or biological half-life of less than 20 days will likely increase the therapeutic window for said agonist in a sequential treatment setting, whereas a DR5 agonist with a longer half-life may cause toxicity. A short-lived DR5 agonist may have similar anti-cancer effect as longer lived DR5 agonists, but have reduced side effects including toxicity.

Said selective DR5 agonist may be a natural or synthetic molecule, a peptide or peptide analogue, or an antibody.

Said natural or synthetic molecule preferably is a low molecular weight molecule of <1 kiloDalton, preferably of 500 Dalton or less. Said molecule preferably shows good absorption in biological systems and is consequently more likely to be a successful drug candidate than a molecule with a molecular weight above 1 kD or even above 500 Dalton (Lipinski et al., 1997. Advanced Drug Delivery Reviews 23: 3-25). Synthetic compound libraries (e.g. LOP AC™, Sigma Aldrich) or natural compound libraries (Specs, TimTec) may be screened to identify said molecules.

Said selective DR5 agonist preferably is an antibody, preferably a human or humanized antibody. Preferred methods for humanizing antibodies include grafting of CDRs (Queen et al., 1989. PNAS 86: 10029; Carter et al., 1992. PNAS 89: 4285; resurfacing (Padlan et. al., 1991. Mol Immunol 28: 489; superhumanization (Tan et. al., 2002. J Immunol 169: 1119), human string content optimization (Lazar et al., 2007. Mol Immunol 44: 1986) and humaneering (Almagro et. al., 2008. Frontiers Biosci 13: 1619). Further preferred methods are described in the published international applications WO2011080350; WO2014033252 and WO2009004065; and in Qu et al., 1999. Clin. Cancer Res. 5: 3095-3100; Ono et al., 1999. Mol. Immunol. 36: 387-395; These methods rely on analyses of the antibody structure and sequence comparison of the non-human and human antibodies in order to evaluate the impact of the humanization process into immunogenicity of the final product.

Said selective DR5 agonist may be a reformatted antibody, in which CDRs from one antibody class are grafted to the frame work regions of another antibody class. Said selective DR5 agonist preferably is a reformatted antibody, in which CDRs from one antibody class are grafted to the frame work regions of another antibody class, preferably grafted to the frame work regions of an IgA or IgA-like antibody. As an alternative, or in addition, a part of an IgD, IgE, IgG, or IgM antibody, for example a variable region, may be fused to an IgA constant region or a part thereof, preferably a human IgA constant region or a part thereof. Said IgA constant region preferably includes at least part of the CD89-interacting domain in the Cα3 domain, including at least amino acid residues L441A442 (Pleass et al., 1999. JBC 274: 23508-23514), preferably a complete Cα3 domain, preferably complete Cα2 and Cα3 domains, preferably a complete constant region (Cα1, Cα2, Cα3), optionally including a hinge region.

As is shown in the examples, the variable region of an anti-DR5 antibody may be linked to the constant region of an IgA heavy chain antibody. The resulting fusion antibody comprises an anti-DR5 variable region fused to a part or a complete constant region of a IgA antibody heavy chain. Said anti-DR5 antibody, or DR5 binding part thereof such as the variable region of said anti-DR5 antibody, may be any one of tigatuzumab (CS-1008), lexatumumab (HGS-ETR2), HGS-TR2J, drozitumab (APOMAB), conatumumab (AMG-655), zaptuzumab (Chen et al., 2017. UBMB Life 69: 735-744), IGM 8444 (Wang et al., 2021. Mol Cancer Therapeutics 20: 2483-2494), CTB006 (Zheng and Shen, 2011. Chin Med Biotechnol 6: 106-110), and LBY135 (Sharma et al., 2014. Invest New Drugs 32: 135-44). Said anti-DR5 antibody, or DR5 binding part thereof, may be an antibody as described in any one of WO 98/51793, WO 2001/83560, WO 2002/94880, WO 2003/54216, WO 2006/83971, WO 2007/22157 or WO 2012/057288, which are all incorporated herein by reference.

Said fusion antibody, comprising an anti-DR5 variable region fused to the constant region of a IgA antibody heavy chain, preferably comprises a multimerization domain, such as a dimerization domain. Said multimerization domain may be any domain that facilitates multimerization such as dimerization of a protein, including a leucine zipper-based dimerization domain, a tetratrico peptide repeat domain, a Bric-à-brac, Tramtrack, and Broad Complex (BTB) domain, an immunoglobulin J chain such as UniProtKB P01591, a C(H)3 domain in the constant region of an antibody IgG heavy chain, or a part or a variant, including a tagged variant, thereof.

In an embodiment, said anti-DR5 IgA or IgA-like antibody comprises a conatumumab variable region, fused to an IgA constant region. Said anti-DR5 IgA or IgA-like antibody preferably comprises the amino acid sequences of SEQ 2 and SEQ 3, preferably of SEQ 2, SEQ 3 and SEQ 4, as indicated herein below.

The invention further provides a selective DR5 agonist, preferably having an in vivo half-life of less than 20 days, for use in a method of selectively killing of senescent cells such as senescent cancer cells.

The provision of a selective DR5 agonist may be combined with the provision of a BRD2 inhibitor. It was found that a BRD2 inhibitor greatly enhances DR5-mediated killing of senescent cells. A BRD2 inhibitor was found to reduce expression of CASP8 And FADD Like Apoptosis Regulator (CFLAR), also termed Cellular FLICE (FADD-like IL-1β-converting enzyme)-inhibitory protein (CFLIP), which acts to inhibit DR5 killing.

Said BRD2 inhibitor may be selected from BRD2 Bromodomain-Interactive Compound (BIC1; 1-(2-(1H-benzimidazol-2-ylsulfanyl)ethyl)-3-methyl-1,3-dihydro-2H-benzimidazole-2-thione), or olinone (2,3,4,5-tetrahydro-5-(4′-acetamidobutyl)-1H-pyrido-[4,3-b]indol-1-one), which are selective for a first BET domain in BRD2; apabetalone (RVX-208; 2-[4-(2-hydroxyethoxy)-3,5-dimethylphenyl]-5,7-dimethoxy-4(3H)-quinazolinone) or ABBV-744 (N-ethyl-4-(2-(4-fluoro-2,6-dimethylphenoxy)-5-(2-hydroxypropan-2-yl)phenyl)-6-methyl-7-oxo-6,7-dihydro-1H-pyrrolo[2,3-c]pyridine-2-carboxamide), which are selective for a second BET domain in BRD2; I-BET 151 (GSK1210151A; 7-(3,5-dimethyl-1,2-oxazol-4-yl)-8-methoxy-1-[(1R)-1-pyridin-2-ylethyl]-3H-imidazo[4,5-c]quinolin-2-one); I-BET 762 (GSK525762; 2-[(4S)-6-(4-chlorophenyl)-8-methoxy-1-methyl-4H-[1,2,4]triazolo[4,3-a][1,4]benzodiazepin-4-yl]-N-ethylacetamide), birabresib (OTX-015; 2-[(9S)-7-(4-chlorophenyl)-4,5,13-trimethyl-3-thia-1,8,11,12-tetrazatricyclo[8.3.0.0^{2,6}]trideca-2(6),4,7,10,12-pentaen-9-yl]-N-(4-hydroxyphenyl)acetamide), TEN-010 (2-[(9S)-7-(4-chlorophenyl)-4,5,13-trimethyl-3-thia-1,8,11,12-tetrazatricyclo[8.3.0.02,6]trideca-2(6),4,7,10,12-pentaen-9-yl]-N-[3-(4-methylpiperazin-1-yl)propyl]acetamide), CPI-203 (2-[(9S)-7-(4-chlorophenyl)-4,5,13-trimethyl-3-thia-1,8,11,12-tetrazatricyclo[8.3.0.02,6]trideca-2(6),4,7,10,12-pentaen-9-yl]acetamide) or pelabresib (CPI-0610; 2-[(4S)-6-(4-chlorophenyl)-1-methyl-4H-[1,2]oxazolo[5,4-d][2]benzazepin-4-yl]acetamide), which do not appear to be selective for a BET domain in BRD2; or NEO2734 (1,3-dimethyl-5-[2-(oxan-4-yl)-3-[2-(trifluoromethoxy)ethyl]benzimidazol-5-yl]pyridin-2-one), which inhibits both BET domains and cAMP response element-binding protein-binding proteins.

In addition, LY294002 (2-morpholin-4-yl-8-phenylchromen-4-one), AZD5153 ((3R)-4-[2-[4-[1-(3-methoxy-[1,2,4]triazolo[4,3-b]pyridazin-6-yl)piperidin-4-yl]phenoxy]ethyl]-1,3-dimethylpiperazin-2-one), MT-1 (2-[(9S)-7-(4-chlorophenyl)-4,5,13-trimethyl-3-thia-1,8,11,12-tetrazatricyclo[8.3.0.0^{2,6}]trideca-2(6),4,7,10,12-pentaen-9-yl]-N-[2-[2-[2-[2-[2-[2-[2-[2-[[2-[(9S)-7-(4-chlorophenyl)-4,5,13-trimethyl-3-thia-1,8,11,12-tetrazatricyclo[8.3.0.0^{2,6}]trideca-2(6),4,7,10,12-pentaen-9-yl]acetyl]amino]ethoxy]ethoxy]ethoxy]ethoxy]ethoxy]ethoxy]ethoxy] ethyl]acetamide), HY-103036 (2-[(4S)-6-(4-chlorophenyl)-1-methyl-8-(1-methylpyrazol-4-yl)-4H-[1,2]oxazolo[5,4-d][2]benzazepin-4-yl]acetamide), HY-43723 ((9S)-7-(4-chlorophenyl)-9-(2-methoxy-2-oxoethyl)-5,13-dimethyl-3-thia-1,8,11,12-tetrazatricyclo[8.3.0.02,6]trideca-2(6),4,7,10,12-pentaene-4-carboxylic acid), HY-13235 (7-(3,5-dimethyl-1,2-oxazol-4-yl)-8-methoxy-1-[(1R)-1-pyridin-2-ylethyl]-3H-imidazo[4,5-c]quinolin-2-one), BETd-246 (4-[(5-cyclopropyl-2-ethylpyrazol-3-yl)amino]-7-(3,5-dimethyl-1,2-oxazol-4-yl)-N-[3-[2-[2-[3-[[2-(2,6-dioxopiperidin-3-yl)-1,3-dioxoisoindol-4-yl]amino]propoxy]ethoxy]ethoxy]propyl]-6-methoxy-9H-pyrimido[4,5-b]indole-2-carboxamide), and MS645 (2-[1-(benzenesulfonyl)-5-methoxyindol-3-yl]-N,N-dimethylethanamine) have been reported to inhibit BET-domain proteins such as BRD2.

Said BRD2 inhibitor may further include a proteolysis-targeting chimeric molecule (PROTAC)-based drug that targets BRD2, preferably is specific for BRD2. Said PROTAC-based drug preferably comprises a single domain antibody, such as a camelid heavy chain only antibody, also termed VHH antibody, or human that recognizes BRD2, preferably specifically recognizes BRD2, which is coupled to a molecule that engages an E3 ubiquitin ligase, preferably coupled to E3 ubiquitin ligase. Said single domain antibody preferably is humanized. Said E3 ubiquitin ligase preferably is a human E3 ubiquitin ligase, also termed Parkinson protein 2 or parkin, having UniProt accession code O60260. Monoclonal anti-BRD2 antibodies are commercially available, for example from HUABIO (Cambridge, MA, USA), ThermoFisher Scientific (Waltham, MA, USA), and Novus Biologicals (Centennial, CO, USA).

### 4.3 Compositions

A selective DR5 agonist for use in a method of treating a patient suffering from a pathology involving senescent cells preferably is provided as a pharmaceutical preparation, comprising one or more pharmaceutically acceptable excipients. Said selective DR5 agonist preferably has a short in vivo half-life of 20 days or less, such as 4-9 days. Said selective DR5 agonist preferably is an antibody, preferably a human or humanized antibody, preferably an human or humanized IgA or IgA-like antibody.

A combination of an inducer of senescence and a selective DR5 agonist for use according to the invention may be provided in one pharmaceutical preparation, or as two or more distinct pharmaceutical preparations.

When provided as a single pharmaceutical preparation, said preparation preferably is a time controlled-release formulation that releases the inducer of senescence in advance of the selective DR5 agonist. Release of the inducer of senescence preferably is at least 24 hours prior to the release of the selective DR5 agonist, preferably 3-7 days.

As is indicated herein above, said selective DR5 agonist may be combined with a BRD2 inhibitor, preferably a selective BRD2 inhibitor. It was found that a BRD2 inhibitor enhances the senolytic effect of a DR5 agonist (see example 2). Said BRD2 inhibitor may be selected from BIC1, olinone, apabetalone, ABBV-744, I-BET 151, I-BET 762, birabresib, TEN-010, CPI-203, pelabresib, NEO2734, LY294002, MT-1, HY-103036, HY-43723, HY-13235, BETd-246 and MS645. Said combination of a selective DR5 agonist and a BRD2 inhibitor may be provided in one pharmaceutical preparation, or as two or more distinct pharmaceutical preparations.

Said single or distinct pharmaceutical preparations may further comprise pharmaceutically acceptable excipients, as is known to a person skilled in the art. For oral administration, a preferred pharmaceutical preparation is provided by a tablet.

Pharmaceutically acceptable excipients include diluents, binders or granulating ingredients, a carbohydrate such as starch, a starch derivative such as starch acetate and/or maltodextrin, a polyol such as xylitol, sorbitol and/or mannitol, lactose such as α-lactose monohydrate, anhydrous α-lactose, anhydrous β-lactose, spray-dried lactose, and/or agglomerated lactose, a sugar such as dextrose, maltose, dextrate and/or inulin, or combinations thereof, glidants (flow aids) and lubricants to ensure efficient tableting, and sweeteners or flavours to enhance taste.

The invention therefore provides a pharmaceutical composition, comprising an inducer of senescence and a selective DR5 agonist, optionally further combined with a BRD2 inhibitor. Said pharmaceutical composition preferably is for use in a method of treating a patient suffering from a tumor, such as a solid tumor, preferably a carcinoma.

The invention further provides a kit of parts, comprising an inducer of senescence and a selective DR5 agonist, and optionally further comprising a BRD2 inhibitor, as a combined preparation for simultaneous, separate or sequential use in the treatment of a tumor in a subject.

For the purpose of clarity and a concise description, features are described herein as part of the same or separate aspects and preferred embodiments thereof, however, it will be appreciated that the scope of the invention may include embodiments having combinations of all or some of the features described.

The invention will now be illustrated by the following examples, which are provided by way of illustration and not of limitation and it will be understood that many variations in the methods described and the amounts indicated can be made without departing from the spirit of the invention and the scope of the appended claims.

### EXAMPLES

### Example 1

### Materials and Methods

Alisertib, Etoposide, CFI-400945, Barasertib, ABT263/Navitoclax, NEO2734 and iBET were purchased from Selleckchem (Houston, TX, USA). Lz-TRAIL was purchased from LSBio (Seattle, WA, USA). Conatumumab was purchased from Creative Biolabs (Shirley, NY, USA). Guide-RNA (gRNA) targeting cFLIP were cloned into Lentiguide-puro (Addgene, Cambridge, MA, USA). shRNA targeting DR4, DR5 were from the TRC shRNA collection (SigmaAldrich, St. Louis, MO, USA).

A549 cells were infected with lentiviral vector Edit-R Inducible Lentiviral Cas9 and doxycycline TRIPZ Inducible Lentiviral shRNA vectors targeting cFLIP (Dharmacon™, Lafayette, CO, USA). Next, Brunello lentiviral whole genome-wide gRNA collection virus (Addgene) was introduced to the A549-iCas9 cells. These infected cells were firstly treated with 0.5 μM alisertib for 7 days to drive them into senescence. Afterwards, these cells were suspended from alisertib then switched to 1 μg/ml doxycycline (DOX) treatment for 10 days. Non-senescent cells were included as the control arm to filter out the straight lethal genes and also treated with doxycycline. Changes in library representation after 10 days doxycycline treatment were determined by Illumina deep-sequencing. Proliferating cells were also taken as the control.

RNA sequencing was performed on A549 cells treated with 0.5 alisertib μM for 1 week, and followed by gene set enrichment analysis (GSEA) of alisertib treated cells versus untreated control for multiple independent NF-KB signaling gene sets. (B) Real-time PCR analysis of DR4, DR5, cFLIP, TRAIL in A549 cells treated with 0.5 μM alisertib, 100 nM CFI-400945 and 2 μM Etoposide. GAPDH served as control.

### Results

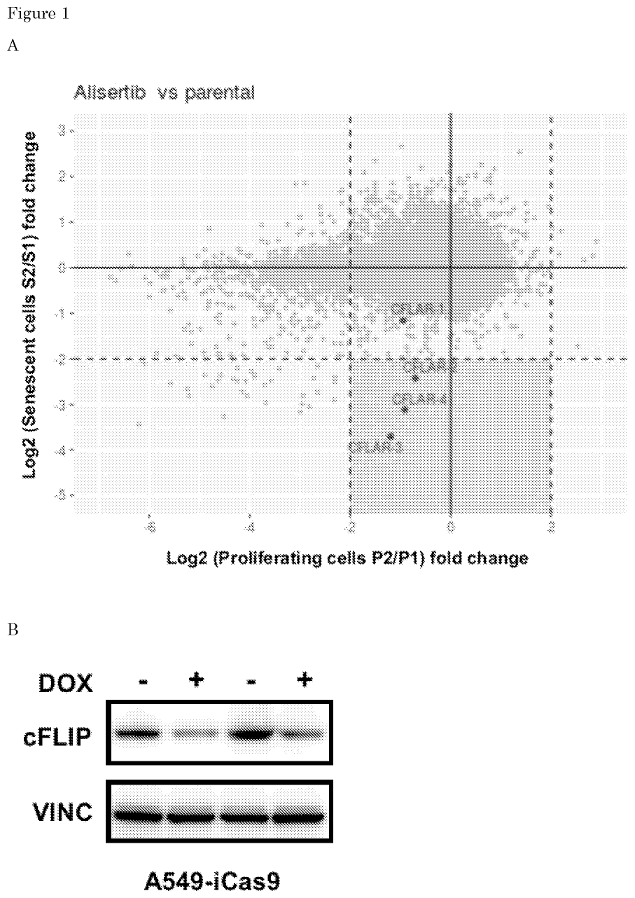

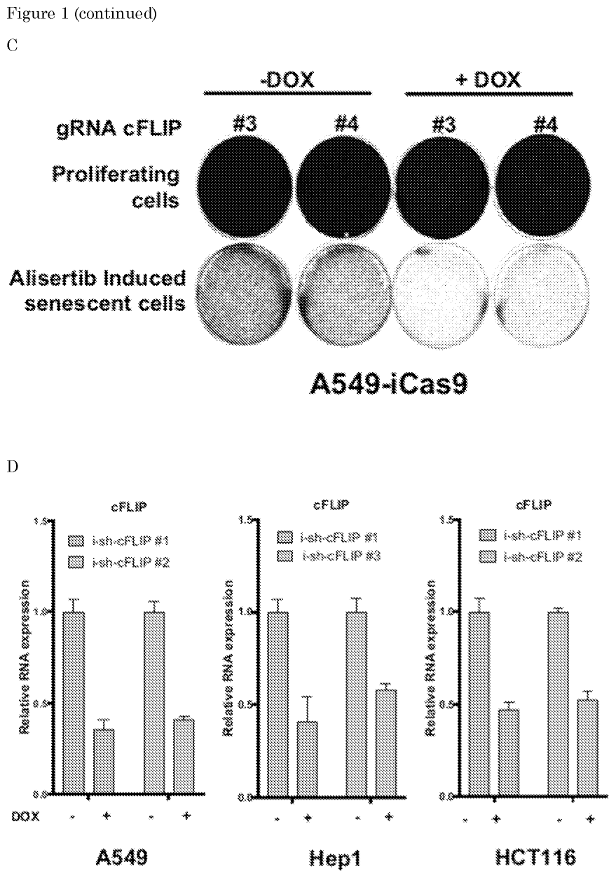

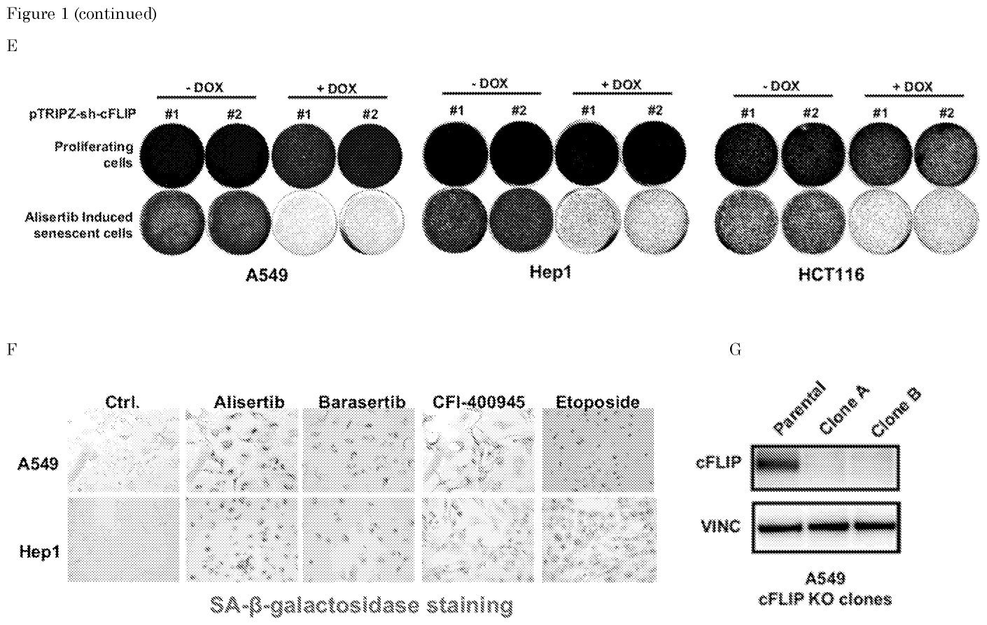

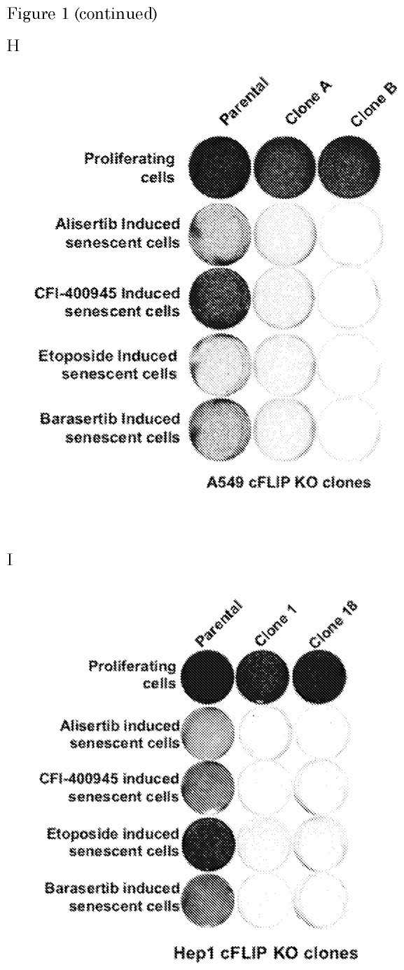

The design of a CRISPR/Cas9 based genetic screen platform to identify genes whose inactivation causes cell death in senescent cancer cells, but not in proliferating counterparts has been described in Wang et al., 2019 (Wang et al., 2019. Nature 574: 268-272). In a first screen, a KRAS mutant lung cancer line A549 was used as screening model and the aurora kinase A inhibitor alisertib as senescence inducer. Using this approach, the CASP8 And FADD Like Apoptosis Regulator (CFLAR) gene was identified (also known as cFLIP), an inhibitor of death receptor mediated apoptosis, as top hit (FIG. 1A). This gene was chosen for further validation. FIGS. 1B and 1C show that polyclonal infection of A549 cells with independent gRNAs targeting cFLIP resulted in the reduction of gene expression and preferential killing of senescent cells. To expand validation to additional cell models, inducible shRNA targeting cFLIP was used in liver cancer cells and colon cancer cells and similar effects were observed (FIGS. 1D and 1E).

In addition, monoclonal cFLIP knockout clones were generated from A549 and Hep1 cell lines. It could again be observed that loss of cFLIP selectively induces cell death in senescent cells. Moreover, the senolytic effect was not only seen in alisertib-induced senescent cells, but also in cells made senescent by other agents, such as PLK4 inhibitor CFI-400945, topoisomerase II inhibitor etoposide and Aurora kinase B inhibitor barasertib (FIGS. 1F-I).

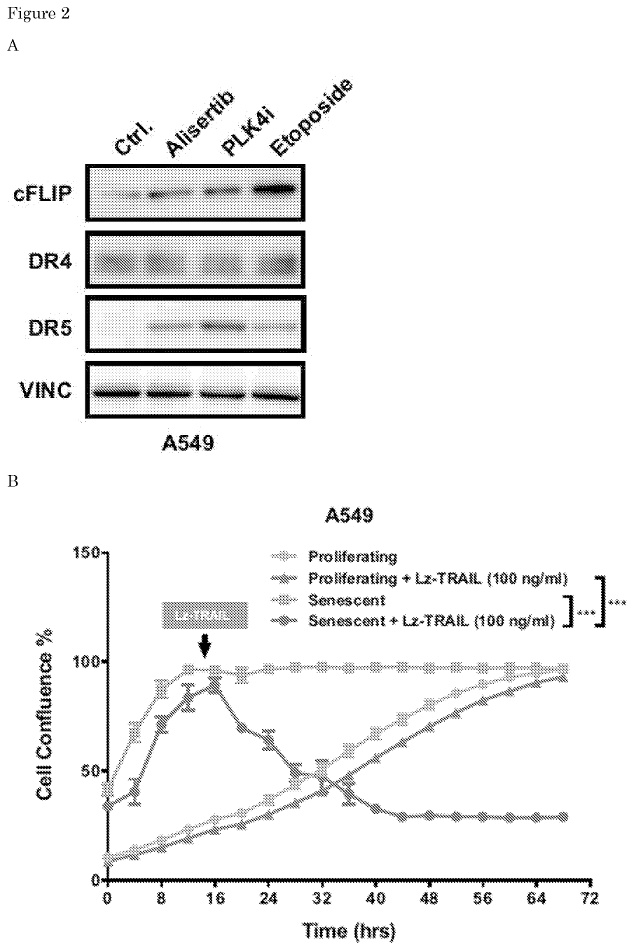

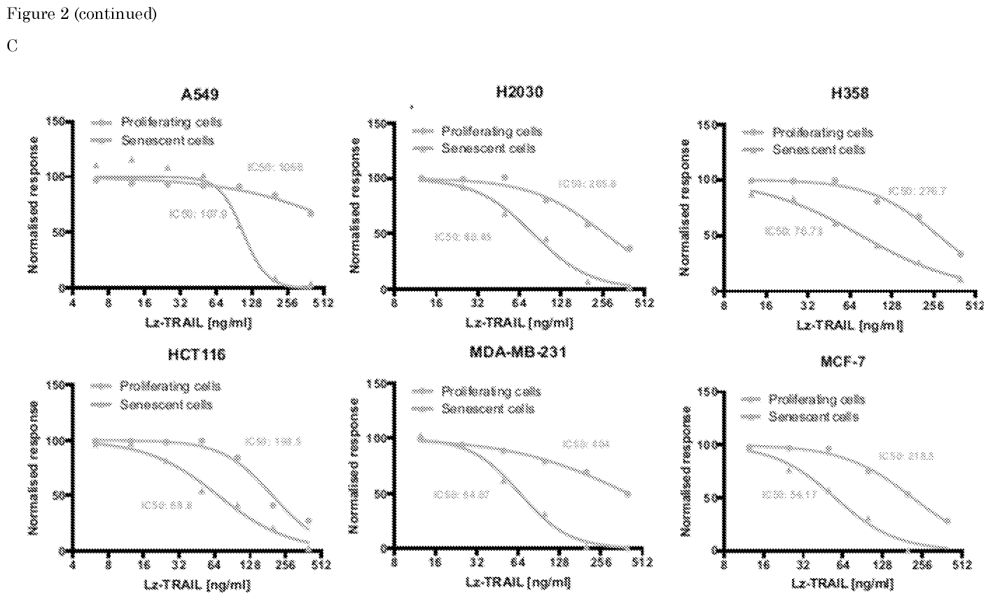

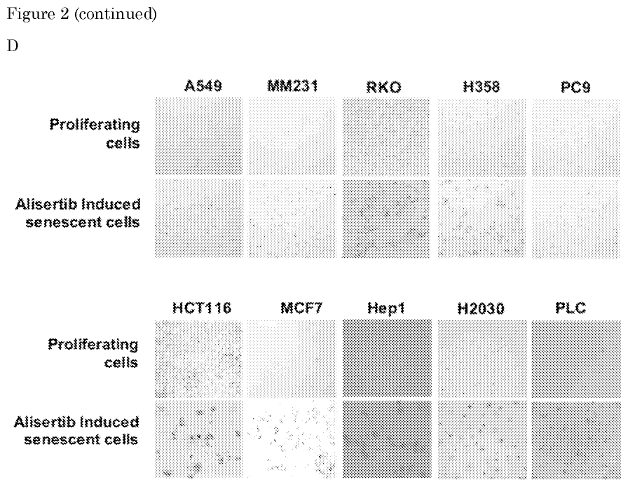

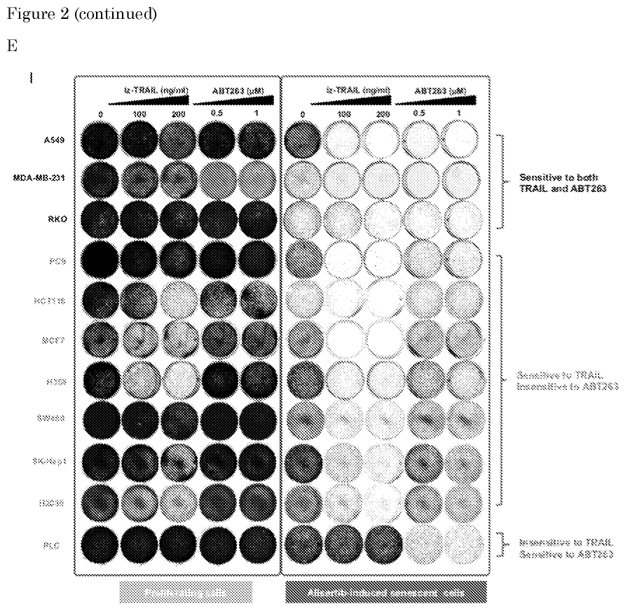

To gain insight into why cFLIP knockout induces senolysis, a transcriptome analysis was performed on the alisertib treated cells using RNA sequencing. It was observed that multiple independent NF-kB signaling signatures were highly enriched in senescent cells, and that cFLIP as a NF-kB target genes became upregulated in the senescent cells (data not shown). To investigate why cFLIP is upregulated in the senescent cells, other components of the death receptor pathway were analyzed. Real-time PCR and western blot analyses showed that the expression of death receptor 5 (DR5) and its ligand TRAIL were highly upregulated in senescent cells (FIG. 2A). To test whether NF-kB signaling is causal in the regulation of cFLIP, DR5 and TRAIL, a critical component of the NF-kB complex, RelA/p65, was suppressed using shRNA. p65 suppression was indeed found to reduce the expression of cFLIP, DR5 and TRAIL (data not shown). These data indicate that senescent cells are primed for apoptotic cell death by upregulation of both TRAIL and DR5, but that these cells are protected from death by cFLIP activation. These data also suggest that there is a dynamic and fine-tuned balance between pro and anti-death receptor signaling to control cell death in these senescent cells. If the balance is disrupted, cell death can be specifically induced in these senescent cells.

To validate this hypothesis, exogenous recombinant TRAIL was added to activate death receptor signaling in senescent cells. FIG. 2B-C show that activation of death receptors DR4 and DR5 with their ligand TRAIL indeed resulted in preferential killing of senescent cells by inducing apoptosis in multiple cell models including lung, breast, colon and liver cancer. Within these cell models, the senolytic effect of TRAIL and ABT-263 was compared. TRAIL was also observed to induce senolysis in cell models that were resistant to ABT-263 (FIGS. 2D and 2E).

This indicates that activation of death receptors as senolytic agent may have a broader application as compared to ABT-263.

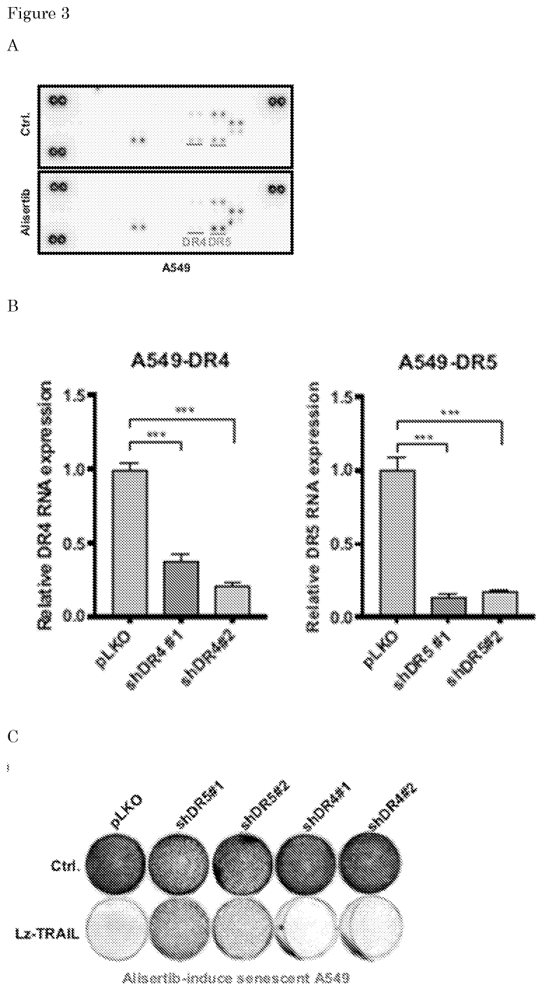

Consistent with the enrichment of multiple independent NF-kB signaling signatures, proteomic analyses showed that only the death receptor TNFRSF10B (DR5), but not TNFRSF10A (DR4), was upregulated in senescent cells (FIG. 3A).

This data is consistent with the RNA sequencing results of a senescent cell line panel, which both showed that TNFRSF10B (DR5) expression is upregulated in alisertib and etoposide-induced senescent cells, but not DR4 (data not shown). To functionally validate this, either DR4 or DR5 expression was suppressed using independent shRNAs and observed that only suppression of DR5 could rescue senescent cells from TRAIL-induced senolysis, but not with DR4 suppression (FIGS. 3B and 3C). These results suggest that activation of DR5 is a significant contributor to sensitizing senescent cells to death receptor pathway agonists. Based on this discovery, it was tested whether a DR5 agonistic antibody, conatumumab, can also preferentially kill senescent cancer cells. This could open an avenue towards therapeutic intervention in the clinic because agonistic DR5 antibody has a higher specificity and better bioavailability than the TRAIL ligand. Consistent with the results obtained with TRAIL, the results show that conatumumab also leads to selective killing of senescent cells in our cell line panel (FIG. 3D-F).

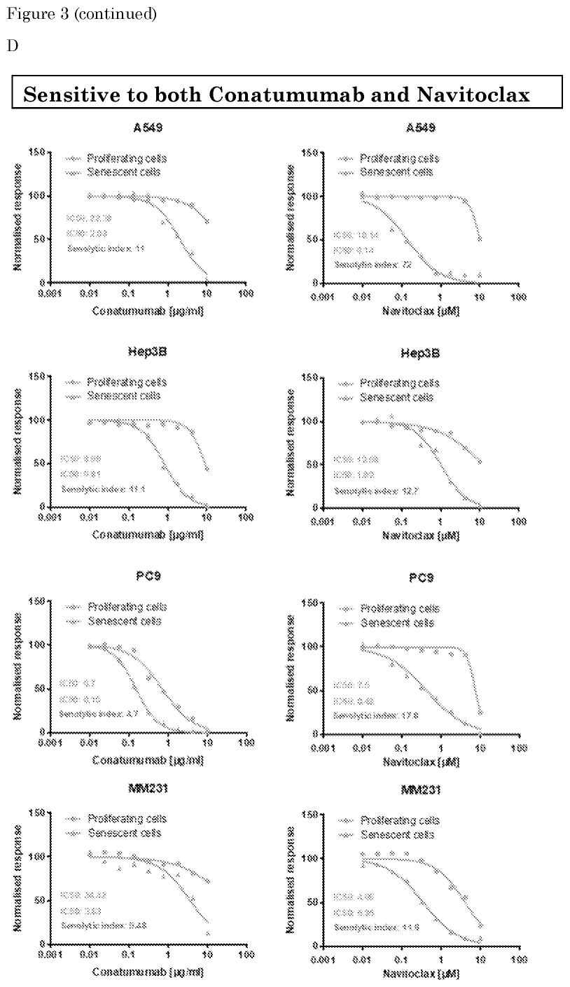

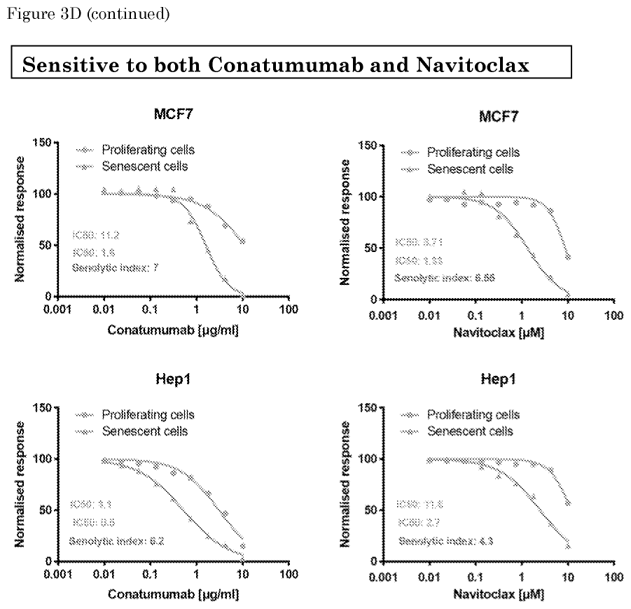

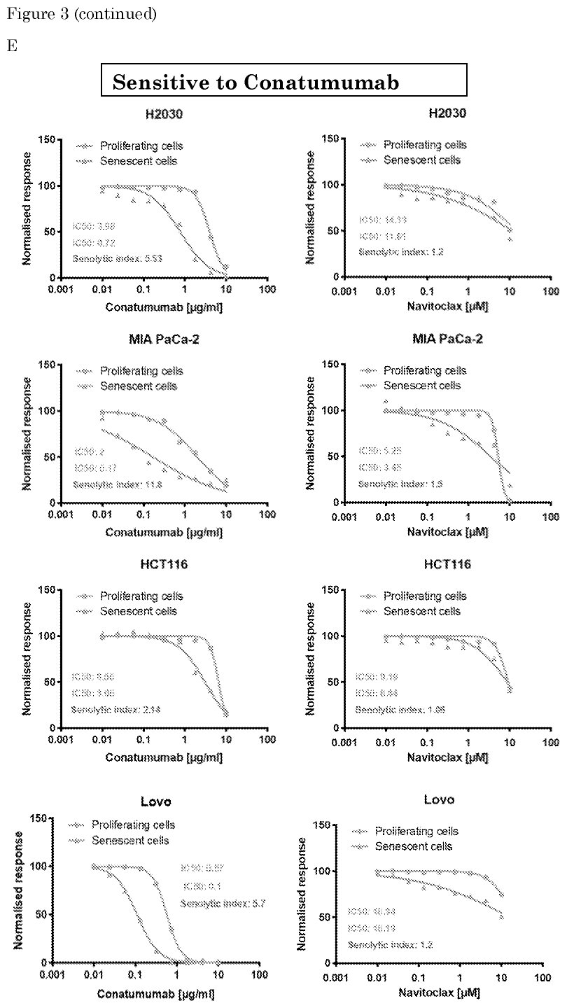

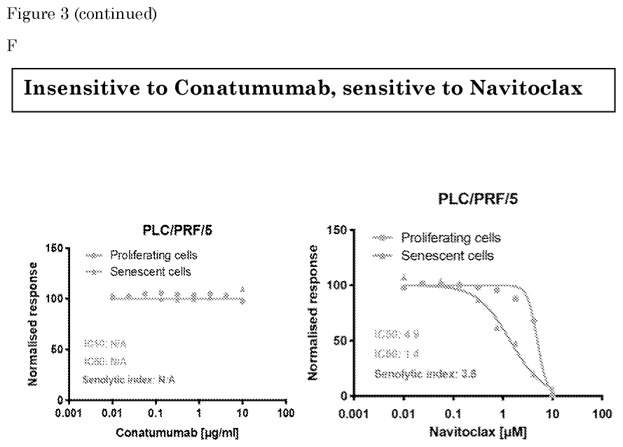

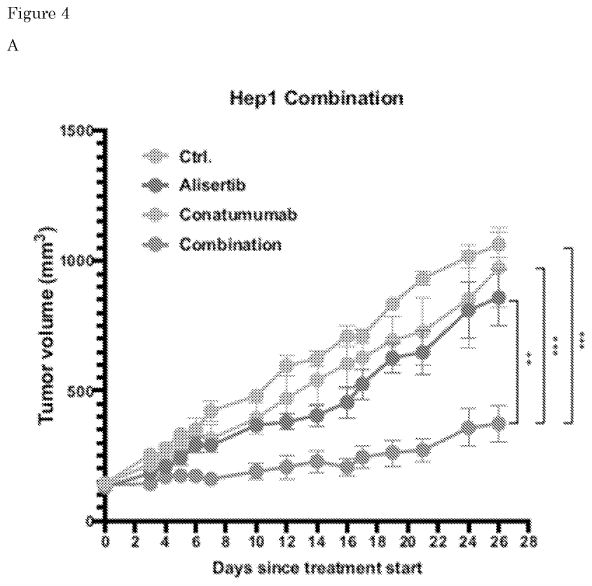

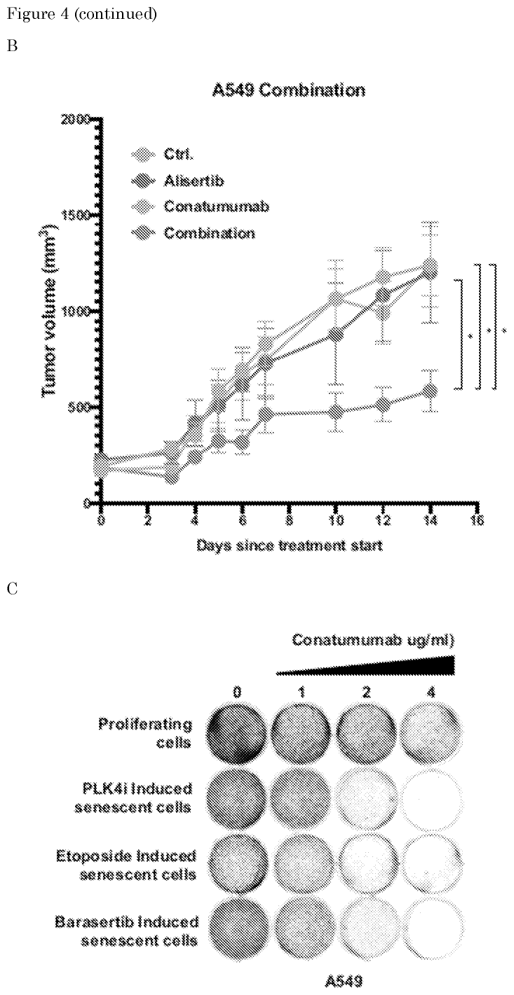

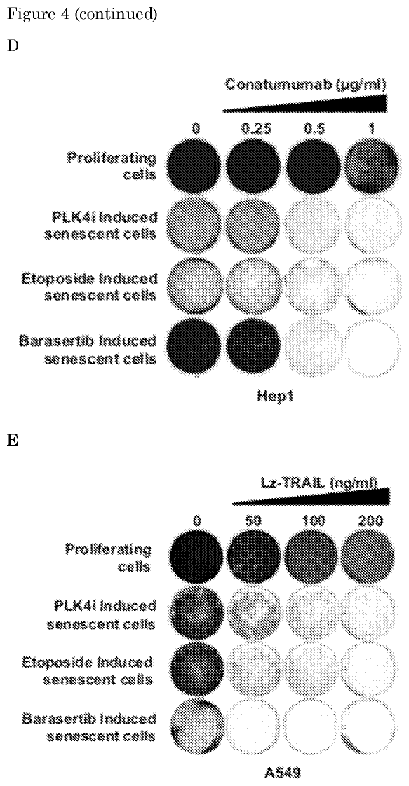

To test the treatment in vivo, we engrafted Hep1 liver cancer cells and A549 lung cancer cells into immunodeficient nude mice. When tumors reached approximately 200 mm³, mice were randomized into different cohorts and treated with vehicle, alisertib, conatumumab and drug combination (due to heterogeneity, senescence is not synchronously induced in the tumors. Therefore, it was decided to use pro-senescence and senolytic drugs in combination). As shown in FIG. 4A-B, treatment with single drugs alisertib and conatumumab resulted in limited tumor growth inhibition. However, treatment with the combination of two drugs resulted in persistent suppression of tumor growth throughout the experiment. To test whether conatumumab and Lz-TRAIL can also be used as a senolytic agent with other senescence inducers, senescent A549 and Hep1 cells were generated with a PLk4 inhibitor, etoposide and barasertib. A strong senolytic effect was again observed when conatumumab or Lz-TRAIL was added to these senescent cells (FIG. 4C-E).

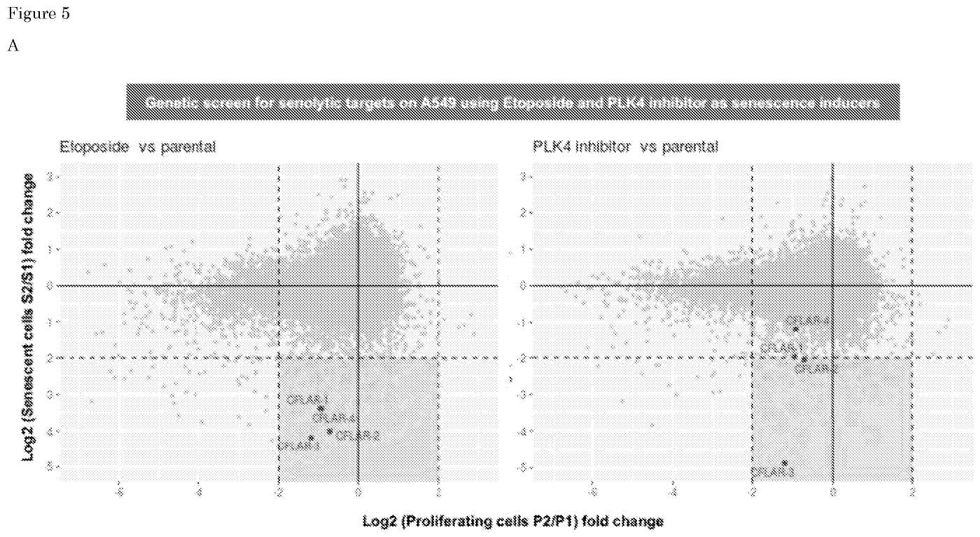

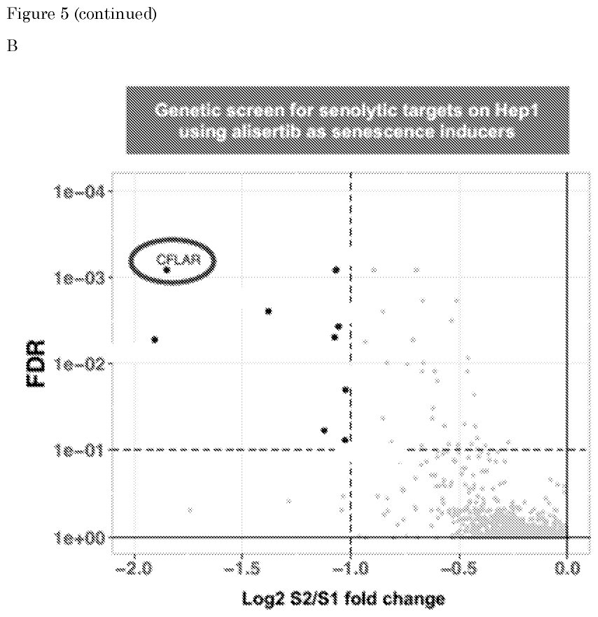

To validate the genetic screen platform further, the senolytic target CRISPR screening platform was further tested using PLK4 inhibitor and etoposide as senescence inducers in A549 cells, and using alisertib in Hep1 cells. Consistent with the first screen, cFLIP was also identified as one of the top hits from the new screens, and other top hits are consistent with the first screen (FIG. 5A-B). These data support that this screening model can be applied broadly with different senescence inducers and different cell models with consistent performance.

### Example 2

### Materials and Methods

### Conatumumab Synergy Screen

PC9 cells were infected with lentiviral vectors containing Brunello lentiviral whole genome-wide gRNA collection virus and CAS9 (Addgene). These infected cells were treated with 0.2 μg/ml conatumumab for 6 days. Non-conatumumab treated cells were included as control arm to filter out direct lethal genes. Changes in library representation after 6 days of conatumumab treatment were determined by Illumina deep-sequencing.

PC9 human lung cancer cells, Panc1 human pancreatic cancer cell line of ductal cell origin, Hep3B human epithelial hepatoma cells, H1975 human epithelial lung cancer cells, TFK bile duct cancer cells, EGI bile duct cancer cells, BJ human foreskin fibroblast cells and Rpe1 human retina pigmented epithelial cells were obtained from ATCC.

### Dose Response Curve

Cells were seeded into 384 well plates. Drugs were added after 24 hours. Cell viability was measured using cell titer blue after 96 hours of drug treatment.

### Colony Formation

Cells were seeded into 6 well plates. Drugs were added after 24 hours. Cells were fixed with 4% paraformaldehyde after 96 hours of drug treatment. The plates were stained with 2 mL 0.5% (w/v) crystal violet in H2O and photographed.

### RNA Sequencing

RNA sequencing was performed on PC9, Hep1, EGI and TFK cells treated with 0.5 μM NEO2734 for 1 week. The expression changes of cFLIP and TNFRSF10B (DR5) were plotted.

### Colony Formation

Cells were treated with 0.5 μM alisertib for 7 days to induce senescence. These cells were seeded into 12 well plates. After 24 hours, the cells were treated with 0.5 μM NEO 2734 and indicated doses of conatumumab for 96 hours. Cells were fixed with 4% paraformaldehyde after 96 hours of drug treatment. The plates were stained with 2 mL 0.5% (w/v) crystal violet in H2O and photographed. Further, colony formation was investigated using 0.25 μM of NEO2734 and 0.125 μg/ml conatumumab on Hep1 cells made senescent by one-week treatment of 0.5 μM alisertib, 1 μM barasertib, 50 nM CFI-400945 or 2 μM Etoposide. Furthermore, colony formation was investigated using 0.25 μM of NEO2734 and 1 μg/ml conatumumab on A549 cells made senescent by one-week treatment of different senescence inducers, namely 0.5 μM PF-06873600, 100 nM doxorubicin or combination of 5 nM trametinib plus 0.5 μM palbociclib. Doxorubicin, PF-06873600, palbociclib and trametinib were purchased from Selleckchem (Houston, TX, USA).

### Results

To test whether DR5 agonist antibody can be combined with other drugs to enhance the drug sensitivity, a CRISPR based genetic screen was performed on a lung cancer cell line PC9 to identify genes whose inactivation result in synergistically killing of the cells upon DR5 activation. Using this approach, a family member of Bromodomain and Extra-Terminal motif (BET) proteins, BRD2 was identified, but not other BET domain proteins such as BRDT, BRD3 or BRD4.

To validate this finding, a BRD2 inhibitor, NEO2734, was used to combine with a DR5 activation antibody (conatumumab) and to test the combination in multiple cancer models, including a pancreatic cancer line of ductal cell origin Panc1, two epithelial hepatoma cell lines Hep3B and Hep1, two bile duct cancer cell lines TFK and EGI, two epithelial cancer lines PC9 and H1975. In addition, two primary cell lines BJ and Rpe1 were used to determine whether this drug combination would be less harmful to healthy cells.

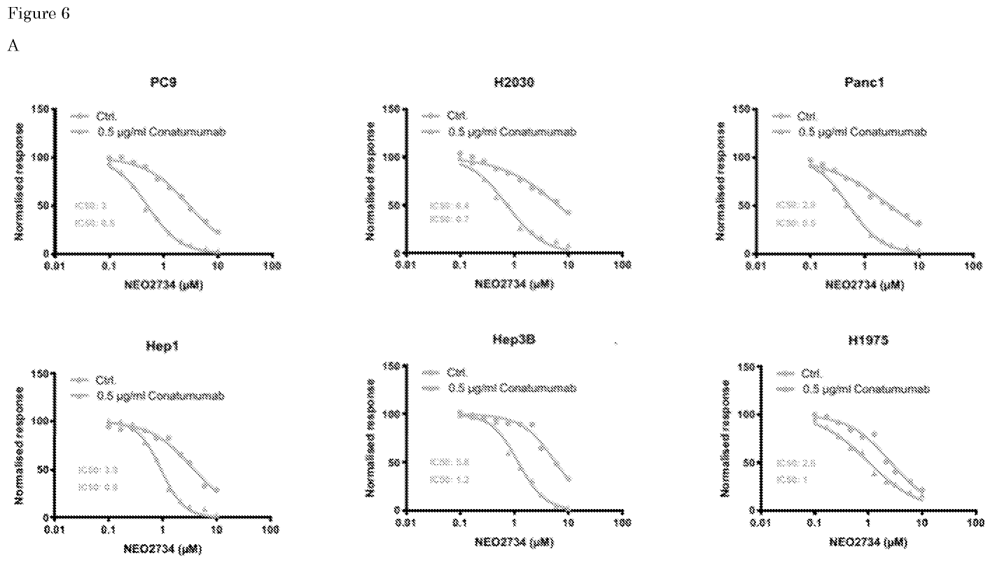

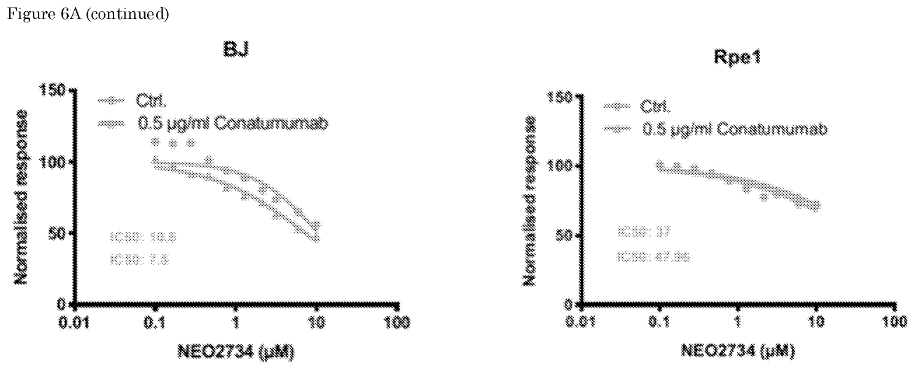

The results of the dose-response curves determined by cell viability using cell titer blue indicated that BRD2 inhibition could indeed enhance the conatumumab responsiveness in the cancer cells, but not in primary cells (FIG. 6A).

To investigate the mechanism of this synergy effect from this drug combination, RNA sequencing was performed on NEO2734 treated cells (using four independent cell models PC9, TFK, EGI and Hep1) to identify genes that might be critical for the DR5 signaling pathway. The RNA sequencing results showed that the death receptor signaling blockade cFLIP (CFLAR) was highly down-regulated upon treatment with a BRD2 inhibitor (FIG. 6B). This might explain why BRD2 inhibition may enhance the responsiveness of conatumumab.

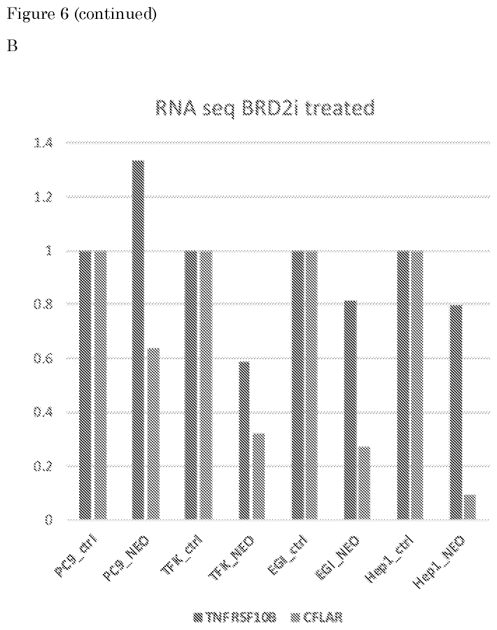

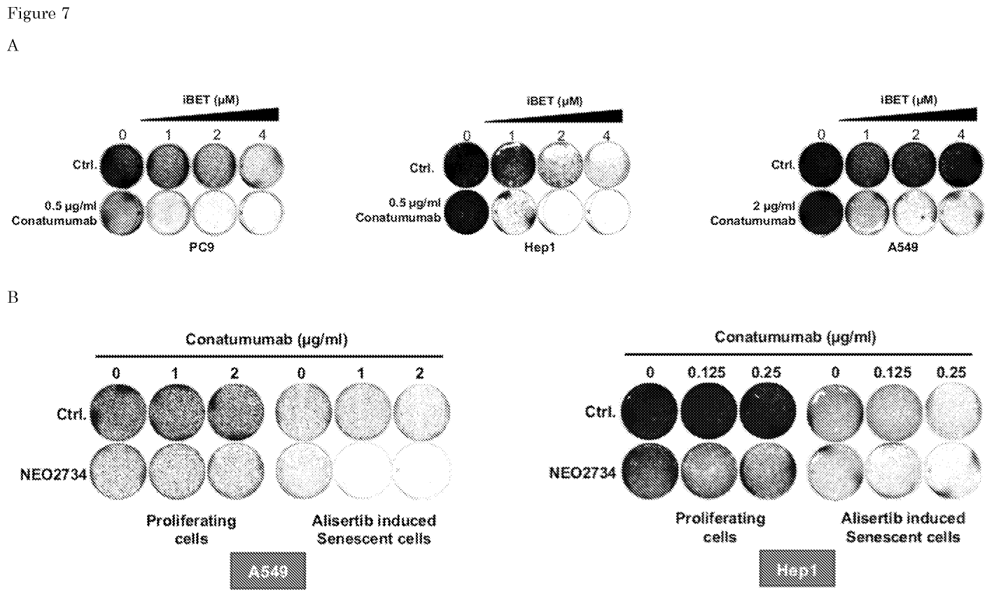

Next, we also tested an alternative BRD2 inhibitor, iBET. The results showed that iBET could also efficiently enhance the responsiveness of conatumumab in the cell models PC9, Hep1 and A549 (FIG. 7A).

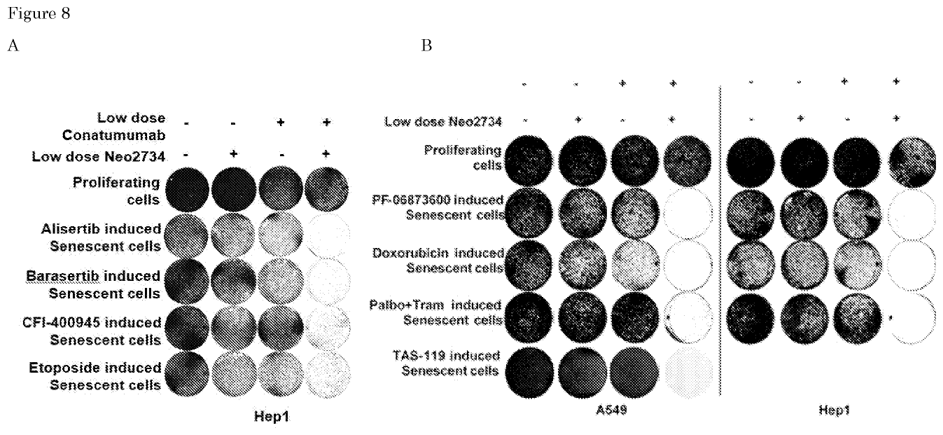

Based on these results, we hypothesized that combining BRD2 inhibition with DR5 activation can lead to a more substantial senolytic effect to kill senescent cancer cells. To test this hypothesis, the dosage of conatumumab that will not lead to a significant impact on the alisertib induced senescent cells was identified by titration experiments in both A549 cells and HEP1 cells (FIG. 7B). Subsequently, these low dosages of conatumumab (1 μg/ml for A549 and 0.125 μg/ml for Hep1) were combined with NEO2734 in the treatment of both proliferating and senescent A549 and Hep1 cells. The result show that indeed BRD2 inhibition did improve the senolytic effect of DR5 activation (FIG. 7B). Moreover, this low dose combination of conatumumab and NEO2734 was validated as a potent senolytic cocktail in multiple therapy-induced senescent models, including barasertib, CFI-400945, Etoposide, doxorubicin, PF-06873600 and the combination of palbociclib and trametinib (FIG. 8A, B).

### Example 3

### Materials and Methods

A549 cells were treated with 20 μg/ml bleomycin for one week. Bleomycin-induced senescent A549 cells are a model for idiopathic pulmonary fibrosis. Colony formation on bleomycin-induced senescent A549 cells treated with 0.25 μM NEO2734 plus 2 μg/ml conatumumab.

### Results

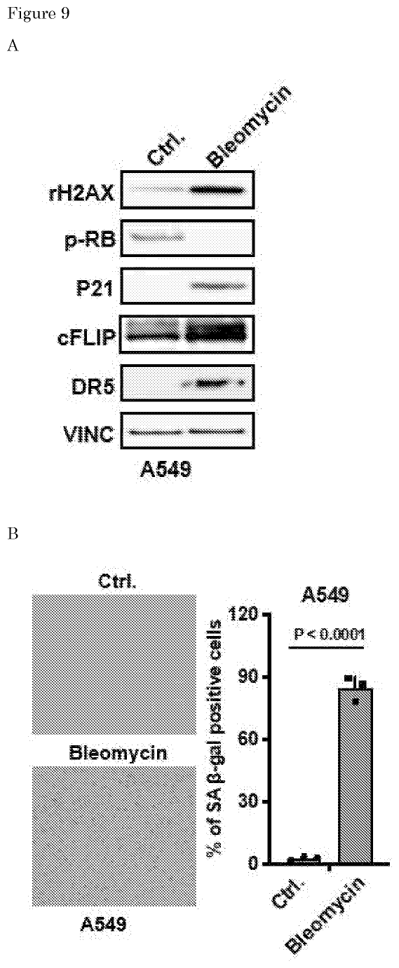

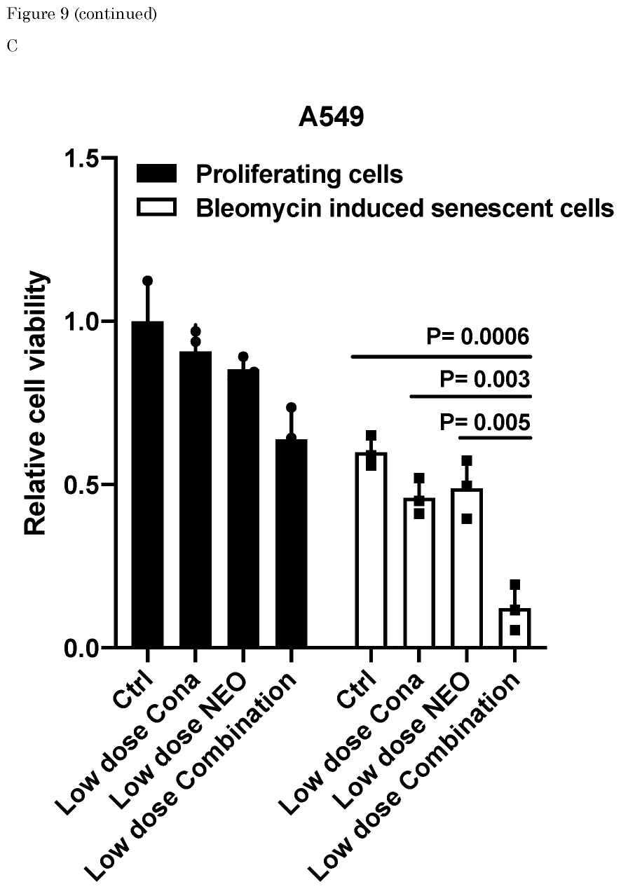

To expand the application of the senolytic cocktail comprising conatumumab and NEO2734, this cocktail was tested in another senescence related disease model, idiopathic pulmonary fibrosis (IPF). As in other studies (Aoshiba et al., 2003. Eur Respir J 22:436-443), A549 cells were used as lung epithelial cells and treated with bleomycin to induce senescence (FIG. 9A, B). The results show that a senolytic cocktail comprising conatumumab and NEO2734 preferentially kills bleomycin-induced senescent cells (FIG. 9C). This data also indicates that this senolytic cocktail may be applied to treat or prevent IPF and COVID-19 induced pulmonary fibrosis.

### Example 4

### Materials and Methods

Proliferating A549 cells were cultured with Senescence-Associated Secretory Phenotype (SASP) medium from alisertib-induced senescent A549 cells or GFP lentivirus containing medium and then treated with 0.25 μM NEO2734 and 0.125 μg/ml IgA-cona-dim.

Antibodies were generated by expression of SEQ 1 and SEQ 3 (IgG1-cona), SEQ 2 and SEQ 3 (IgA-cona), or SEQ 2, SEQ 3 and SEQ 4 (IgA-cona-dim) as described (Beyer et al., 2009. J Immunol Methods 346: 26-37), by employing the nucleotide sequences SEQ 5 and SEQ 6 (IgG1-cona), SEQ 6 and SEQ 7 (IgA-cona), or SEQ 6, SEQ 7 and SEQ 8 (IgA-cona-dim). SEQ 4 depicts a sequence of an immunoglobulin J chain, which links two or more IgA monomer units.

### Results

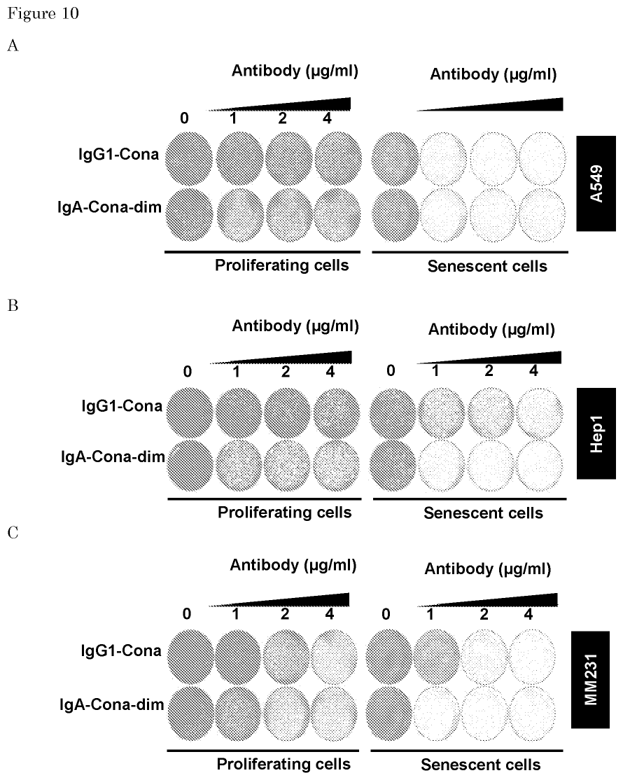

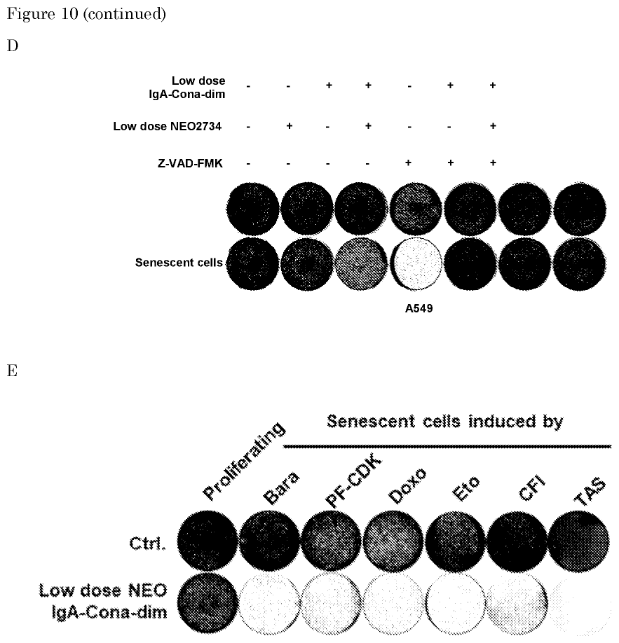

Given the notion that conatumumab kills senescent cells in 24 hours in vitro, the relatively long serum half-life of IgG antibodies (10-21 days) may not be required to obtain efficient cell killing. In addition, prolonged exposure of normal cells to a DR5 agonistic antibody may cause toxicity. We therefore wished to address the fundamental question whether short-lived IgA DR5 agonistic antibodies can have similar senolytic effects as long lived IgG antibodies, but potentially have reduced toxicity in vivo due to the shorter half-life. IgA antibodies have a much shorter half-life in humans (3-6 days) and the same is seen in mice (Leusen, 2015. Mol Immunol 68: 35-39). To test this hypothesis and validate DR5 as a suitable target for IgA antibodies, we generated conatumumab (IgG1-cona) and the same variable region antibody, but linked to the constant region of an IgA heavy chain. See SEQ 1, SEQ 2 and SEQ 3. Since DR5 activation requires multimerization of the receptor, we also generated a naturally occurring dimeric form of IgA-conatumumab (IgA-cona-dim), by co-expressing SEQ 4, to ask if this dimeric form is more active in causing cell death than the monomeric form. First, we tested side-by-side senolytic efficacy of IgG1 and IgA-cona-dim forms of conatumumab on the alisertib-induced senescent A549, Hep1 and MM231 cells. We found that IgA-cona-dim antibody is the most potent DR5 agonist to eliminate senescent cells (FIG. 10A-C). Next, we also reduced the dose of IgA-cona-dim and combined it with a low dose NEO2734 as a senolytic cocktail. We observed that the low dose cocktail also displayed a robust senolytic efficacy. Moreover, the pan-caspase inhibitor Z-VAD-FMK could fully rescue the cells from senolysis. This indicates that apoptosis is the dominant form of cell death, which contributes to the IgA-cona-dim plus NEO2734 mediated senolysis (FIG. 10D). We further validated the senolytic efficacy of a low dose cocktail in multiple senescent models induced by independent senescence inducers, including barasertib, PF-06873600, doxorubicin, etoposide and CFI-400941 (See FIG. 10E).

SEQ 1
Amino acid sequence heavy chain of conatumumab IgG1.
MACPGFLWALVISTCLEFSMAQVQLQESGPGLVKPSQTLSLTCTVSGGSISSGD
YFWSWIRQLPGKGLEWIGHIHNSGTTYYNPSLKSRVTISVDTSKKQFSLRLSSVT
AADTAVYYCARDRGGDYYYGMDVWGQGTTVTVSSASTKGPSVFPLAPSSKSTS
GGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYT
LPPSRDELTKNQVSLTCLVKGFYPSDIAVEWESNGQPENNYKTTPPVLDSDGSF
FLYSKLTVDKSRWQQGNVFSCSVMHEALHNHYTQKSLSLSPGK-.
SEQ 2
Amino acid sequence heavy chain of conatumumab IgA2.
MACPGFLWALVISTCLEFSMAQVQLQESGPGLVKPSQTLSLTCTVSGGSISSGD
YFWSWIRQLPGKGLEWIGHIHNSGTTYYNPSLKSRVTISVDTSKKQFSLRLSSVT
AADTAVYYCARDRGGDYYYGMDVWGQGTTVTVSSASPTSPKVFPLSLDSTPQD
GNVVVACLVQGFFPQEPLSVTWSESGQNVTARNFPPSQDASGDLYTTSSQLTLP
ATQCPDGKSVTCHVKHYTNPSQDVTVPCPVPPPPPCCHPRLSLHRPALEDLLLG
SEANLTCTLTGLRDASGATFTWTPSSGKSAVQGPPERDLCGCYSVSSVLPGCAQ
PWNHGETFTCTAAHPELKTPLTANITKSGNTFRPEVHLLPPPSEELALNELVTL
TCLARGFSPKDVLVRWLQGSQELPREKYLTWASRQEPSQGTTTFAVTSILRVAA
EDWKKGDTFSCMVGHEALPLAFTQKTIDRLAGKPTHVNVSVVMAEVDGTCY.
SEQ 3
Amino acid sequence kappa light chain of conatumumab.
MACPGFLWALVISTCLEFSMAEIVLTQSPGTLSLSPGERATLSCRASQGISRSYL
AWYQQKPGQAPSLLIYGASSRATGIPDRFSGSGSGTDFTLTISRLEPEDFAVYYC
QQFGSSPWTFGQGTKVEIKRTVAAPSVFIFPPSDEQLKSGTASVVCLLNNFYPR
EAKVQWKVDNALQSGNSQESVTEQDSKDSTYSLSSTLTLSKADYEKHKVYACE
VTHQGLSSPVTKSFNRGEC.
SEQ 4
Amino acid sequence J chain precursor.
MKNHLLFWGVLAVFIKAVHVKAQEDERIVLVDNKCKCARITSRIIRSSEDPNED
IVERNIRIIVPLNNRENISDPTSPLRTRFVYHLSDLCKKCDPTEVELDNQIVTATQ
SNICDEDSATETCYTYDRNKCYTAVVPLVYGGETKMVETALTPDACYPDLESRG
PFEQKLISEEDLNMHTGHHHHHH.
SEQ 5
Nucleotide sequence heavy chain of conatumumab IgG1.
atggcctgtcctggatttctgtgggccctcgtgatctctacctgcctggaattcagcatggcccaggttcagctgcaagag
tctggacctggcctggtcaagccctctcagaccctgtctctgacctgtacagtgtccggcggctccatctcttccggcgact
acttctggtcctggatcagacagctgcccggcaaaggcctggaatggatcggccacatccacaactctggcaccacctac
tacaaccccagcctgaagtccagagtgaccatctccgtggacacctccaagaagcagttctccctgcggctgtcctctgtg
accgctgctgataccgccgtgtactactgcgccagagatagaggcggcgattactactacggcatggacgtgtggggcc
agggcacaacagtgacagtctcttccgctagcaccaagggaccctctgtgtttcctctggctccctccagcaagtctacct
ctggtggaacagctgccctgggctgcctggtcaaggattactttcctgagcctgtgaccgtgtcctggaactctggcgctc
tgacatctggcgtgcacacctttccagctgtgctgcagtcctccggcctgtactctctgtcctctgtcgtgaccgtgccttcc
agctctctgggcacccagacctacatctgcaatgtgaaccacaagccttccaacaccaaggtggacaagaaggtggaa
cccaagtcctgcgacaagacccacacctgtcctccatgtcctgctccagaactgctcggcggaccttccgtgttcctgtttc
ctccaaagcctaaggacaccctgatgatctctcggacccctgaagtgacctgcgtggtggtggatgtgtctcacgaggac
ccagaagtgaagttcaattggtacgtggacggcgtggaagtgcacaacgccaagaccaagcctagagaggaacagt
acaactccacctacagagtggtgtccgtgctgaccgtgctgcaccaggattggctgaacggcaaagagtacaagtgca
aggtgtccaacaaggccctgcctgctcctatcgaaaagaccatctccaaggccaagggccagcctagggaaccccagg
tttacaccttgcctccatctcgggacgagctgaccaagaaccaggtgtccctgacctgtctcgtgaagggcttctacccct
ccgacattgccgtggaatgggagtctaatggccagcctgagaacaactacaagacaacccctcctgtgctggactccga
cggctcattcttcctgtactccaagctgacagtggacaagtccagatggcagcagggcaacgtgttctcctgctccgtgat
gcacgaggccctgcacaatcactacacacagaagtccctgtctctgtcccctggcaagtaa.
SEQ 6
Nucleotide sequence heavy chain of conatumumab IgA2.
atggcctgtcctggatttctgtgggccctcgtgatctctacctgcctggaattcagcatggccCAGGTTCAGCTG
CAAGAGTCTGGACCTGGCCTGGTCAAGCCCTCTCAGACCCTGTCTCTGACCTG
TACAGTGTCCGGCGGCTCCATCTCTTCCGGCGACTACTTCTGGTCCTGGATCA
GACAGCTGCCCGGCAAAGGCCTGGAATGGATCGGCCACATCCACAACTCTGG
CACCACCTACTACAACCCCAGCCTGAAGTCCAGAGTGACCATCTCCGTGGACA
CCTCCAAGAAGCAGTTCTCCCTGCGGCTGTCCTCTGTGACCGCTGCTGATACC
GCCGTGTACTACTGCGCCAGAGATAGAGGCGGCGATTACTACTACGGCATGG
ACGTGTGGGGCCAGGGCACAACAGTGACAGTCTCTTCCGCTAGCCCTACCTCT
CCTAAGGTGTTCCCTCTGAGCCTGGACAGCACCCCTCAGGATGGAAATGTGGT
GGTGGCCTGTCTGGTGCAGGGATTCTTCCCACAAGAGCCCCTGTCCGTGACTT
GGAGCGAGTCTGGACAGAACGTGACCGCCAGAAACTTCCCACCTTCTCAGGAC
GCCTCTGGCGACCTGTACACCACCTCTTCTCAGCTGACCCTGCCTGCCACACA
GTGCCCTGATGGCAAGTCTGTGACCTGCCACGTGAAGCACTACACCAATCCTA
GCCAGGACGTGACCGTGCCTTGTCCTGTTCCTCCTCCACCTCCTTGCTGTCAC
CCTCGGCTGTCTCTGCACAGACCCGCTCTGGAAGATCTGCTGCTGGGCTCTGA
GGCCAACCTGACATGTACCCTGACCGGCCTGAGAGATGCTTCTGGCGCCACCT
TTACCTGGACACCTTCCAGCGGAAAGTCCGCTGTTCAGGGACCTCCTGAGAGG
GACCTGTGCGGCTGTTACTCTGTGTCCTCTGTGCTGCCTGGCTGTGCCCAGCC
TTGGAATCACGGCGAGACATTCACCTGTACCGCTGCTCACCCCGAGCTGAAAA
CCCCTCTGACCGCCAACATCACCAAGTCCGGCAACACCTTCCGGCCTGAAGTG
CATCTGCTGCCTCCACCTTCCGAGGAACTGGCCCTGAATGAGCTGGTCACCCT
GACCTGTCTGGCCAGGGGCTTTAGCCCTAAGGACGTGCTCGTTAGATGGCTGC
AGGGCTCCCAAGAGCTGCCCAGAGAGAAGTATCTGACCTGGGCCTCTCGGCA
AGAGCCATCTCAGGGCACCACAACCTTTGCCGTGACCAGCATCCTGAGAGTGG
CCGCCGAAGATTGGAAGAAGGGCGACACCTTCAGCTGCATGGTCGGACATGA
AGCCCTGCCTCTGGCTTTCACCCAGAAAACCATCGACAGACTGGCCGGCAAGC
CCACACATGTGAATGTGTCTGTGGTCATGGCCGAGGTGGACGGCACCTGTTAT
taa.
SEQ 7
Nucleotide sequence kappa light chain of conatumumab.
ATGGCCTGTCCTGGATTTCTGTGGGCCCTCGTGATCTCTACCTGCCTGGAATT
CAGCATGGCCGAGATCGTGCTGACCCAGTCTCCTGGCACACTGTCACTGTCTC
CAGGCGAGAGAGCTACCCTGTCCTGTAGAGCTTCCCAGGGCATCTCCAGATCC
TACCTGGCCTGGTATCAGCAGAAGCCTGGACAGGCTCCCAGCCTGTTGATCTA
CGGCGCTTCTTCCAGAGCCACAGGCATCCCTGACAGATTCTCCGGCTCTGGCT
CTGGCACCGACTTCACCCTGACCATCAGCAGACTGGAACCCGAGGACTTCGCC
GTGTACTACTGTCAGCAGTTCGGCTCCTCTCCTTGGACCTTTGGCCAGGGCAC
CAAGGTGGAAATCAAGCGGACAGTGGCCGCTCCTTCCGTGTTCATCTTCCCAC
CTTCCGACGAGCAGCTGAAGTCCGGCACAGCTAGCGTGGTCTGCCTGCTGAAC
AACTTCTACCCTCGGGAAGCCAAGGTGCAGTGGAAGGTGGACAATGCCCTGC
AGTCCGGCAACTCCCAAGAGTCTGTGACCGAGCAGGACTCCAAGGACAGCAC
CTACAGCCTGTCCTCCACACTGACCCTGTCCAAGGCCGACTACGAGAAGCACA
AGGTGTACGCCTGCGAAGTGACCCATCAGGGCCTGTCTAGCCCTGTGACCAAG
TCTTTCAACCGGGGCGAGTGTTAA.
SEQ 8
Nucleotide sequence J chain precursor.
ATGAAGAACCATTTGCTTTTCTGGGGAGTCCTGGCGGTTTTTATTAAGGCTGT
TCATGTGAAAGCCCAAGAAGATGAAAGGATTGTTCTTGTTGACAACAAATGTA
AGTGTGCCCGGATTACTTCCAGGATCATCCGTTCTTCCGAAGATCCTAATGAG
GACATTGTGGAGAGAAACATCCGAATTATTGTTCCTCTGAACAACAGGGAGAA
TATCTCTGATCCCACCTCACCATTGAGAACCAGATTTGTGTACCATTTGTCTGA
CCTCTGTAAAAAATGTGATCCTACAGAAGTGGAGCTGGATAATCAGATAGTTA
CTGCTACCCAGAGCAATATCTGTGATGAAGACAGTGCTACAGAGACCTGCTAC
ACTTATGACAGAAACAAGTGCTACACAGCTGTGGTCCCACTCGTATATGGTGG
TGAGACCAAAATGGTGGAAACAGCCTTAACCCCAGATGCCTGCTATCCTGACC
TCGAGTCTAGAGGGCCCTTCGAACAAAAACTCATCTCAGAAGAGGATCTGAAT
ATGCATACCGGTCATCATCACCATCACCATTGA.

1. A method of treating a patient having a tumor, said method comprising administering of an inducer of senescence, in combination with a selective Death Receptor 5 (DR5) to the patient, optionally whereby said tumor is not a melanoma, wherein the selective DR5 agonist has an in vivo half-life of less than 20 days.

2. The method of claim 1 wherein the inducer of senescence is at least one of chemotherapy, ionizing radiation, a CDK4/6 inhibitor, a polo-like kinase 4 (PLK4) inhibitor, a topoisomerase II inhibitor, and an aurora kinase B inhibitor.

3. The method of claim 1, wherein the inducer is palbociclib, alisertib, TAS-119, PF-06873600, CFI-400945, etoposide, doxorubicin, barasertib, or a combination thereof.

4. The method of claim 1, wherein the inducer of senescence and the selective Death Receptor 5 (DR5) agonist are provided sequentially to the patient.

5. The method of claim 1, wherein the selective Death Receptor 5 (DR5) agonist is combined with a Bromodomain Containing 2 (BRD2) inhibitor.

6. The method of claim 1, wherein the selective DR5 agonist is an antibody and wherein the antibody is, a human or humanized antibody.

7. The method of claim 1, wherein the selective DR5 agonist is an antibody, and wherein the antibody is a human or humanized IgA or IgA-like antibody.

8. The method of claim 1, wherein the tumor is a solid tumor selected from the group consisting of lung cancer, breast cancer, colorectal cancer and liver cancer.

9. A selective Death Receptor 5 (DR5) agonist, wherein the selective DR5 agonist is an antibody, and wherein the antibody is a human or humanized IgA or IgA-like antibody, having an in vivo half-life of less than 20 days.

10. A method of treating a patient suffering from a pathology involving senescent cells, said method comprises administering to the patient an effective amount of the selective DR5 agonist of claim 9.

11. A pharmaceutical composition, comprising the selective DR5 agonist of claim 9, optionally further comprising a BRD2 inhibitor.

12. A pharmaceutical composition, comprising an inducer of senescence and a selective Death Receptor 5 (DR5) agonist having an in vivo half-life of less than 20 days, optionally further comprising a BRD2 inhibitor.

13. A method of treating a patient having a tumor, said method comprising administering to the patient an effective amount of the pharmaceutical composition of claim 12.

14. A method of treating a patient having a tumor comprising administering to the patient an effective amount of an inducer of senescence followed by administering a selective DR5 agonist having an in vivo half-life of less than 20 days, optionally in combination with a BRD2 inhibitor.

15. The method of claim 14, wherein the selective DR5 agonist, optionally in combination with a BRD2 inhibitor, is provided at least 24 hours following the inducer of senescence.

16. The method of claim 14, wherein the inducer of senescence, in combination with a selective Death Receptor 5 (DR5) agonist and, optionally, a BRD2 inhibitor, is provided intermittently to the patient, every other day or every other week.

17. The method of claim 14, wherein the tumor is a solid tumor selected from the group consisting of lung cancer, breast cancer, colorectal cancer and liver cancer.

18. A method of selectively killing of senescent cancer cells comprising contacting the senescent cancer cells with a selective DR5 agonist having an in vivo half-life of less than 20 days.

19. A method of treating a patient having a pathology involving senescent cells comprising
administering an effective amount of the selective DR5 agonist of claim 9 to the patient,
optionally in combination with a BRD2 inhibitor.
